SEQUENCE LISTING (54 sequences)

SEQ ID NO: 1 - DNA, 121 bp, Homo sapiens
ccgcgaacagcgccgccgaacttcccaacaggcagggagagggccgatccgggctggcacrccacgtccccggggtctcagtccctgggtcaacgcagagaggctcgcggtccgcggtgct

SEQ ID NO: 2 - DNA, 121 bp, Homo sapiens
ggaggcgaccggggcgcctctgctccggagggggcagaggagggagggagcgccgggcacmgcgcccggtatctttctgcgcttgactggccgggaggaagggggtgacactggggcaccc

SEQ ID NO: 3 - DNA, 121 bp, Homo sapiens
cctcatccagacgctccgctagtgcagacaggagcgcgcagtggccccggctcgccgcgcyatggagcggatccccagcgcgcaaccaccccccgcctgcctgcccaaagcaccgggactg

SEQ ID NO: 4 - DNA, 121 bp, Homo sapiens
  misc_feature (61)..(61): n is a, c, g, or t
caggatgtaccctgcccacatgtaccaagtgtacaagtcaagacggggaataaagcggagngaggacagcaaggtaagcaagtgcacccctagggaccctgcgctcagcccctcgcgcgcg

SEQ ID NO: 5 - DNA, 121 bp, Homo sapiens
agacaggtggcacccgtttttttaaattatttaattatatatatatattatatatatatayacacacacacacacacacacatatatgagtgtatctgtctatctttttaaaagcatgacc

SEQ ID NO: 6 - DNA, 105 bp, Homo sapiens
  misc_feature (61)..(62): AT is a repeat of 4-7 units;
aatgcctccaaagacaggtggcacccgtttttttaaattatttaattatatatatatattatacatatatgagtgtatctgtctatctttttaaaagcatcacct

SEQ ID NO: 7 - DNA, 121 bp, Homo sapiens
atcagacccagctcccaaagtgatggacttcaaggaaaaacccagctctccggccaaaggytcggaaggtcctgggaaaaactgcgtgccagtcatccagcggactttcgctcactcgagt

SEQ ID NO: 8 - DNA, 121 bp, Homo sapiens
actgcgtgccagtcatccagcggactttcgctcactcgagtggggagcagagcggcagcgrcacggacacagacagtggctatggaggagaatcggagaagggcgacttgcgcagtgagca

SEQ ID NO: 9 - DNA, 122 bp, Homo sapiens
  misc_feature (61)..(62): AT is a repeat of 12-22 units;
ctgcaagattgttgcattgtgtatactgagataatctgaggcatggagagcagattcagggtatgtgcgtgtgcgtgcacatgtgtgcctgcgtgttggtataggactttaaagctcctttt

SEQ ID NO: 10 - DNA, 121 bp, Homo sapiens
ctttaaagctccttttggcatagggaagtcacgaaggattgcttgacatcaggagacttgsgggggattgtagcagacgtctgggcttttccccacccagagaatagcccccttcgataca

SEQ ID NO: 11 - DNA, 121 bp, Homo sapiens
aagctccttttggcatagggaagtcacgaaggattgcttgacatcaggagacttggggggkattgtagcagacgtctgggcttttccccacccagagaatagcccccttcgatacacatca

SEQ ID NO: 12 - DNA, 121 bp, Homo sapiens
tccttttggcatagggaagtcacgaaggattgcttgacatcaggagacttgggggggattrtagcagacgtctgggcttttccccacccagagaatagcccccttcgatacacatcagctg

SEQ ID NO: 13 - DNA, 121 bp, Homo sapiens
catagcacggtagtggtttggggaggtttccgcaggtctgctccccacccctgcctcggargaataaagagaatgtagttccctactcaggctttcgtagtgattagcttactaaggaact

SEQ ID NO: 14 - DNA, 121 bp, Homo sapiens
caacggcatatggagtgtccttattgctaaaaaggattccgtctccttcaaagaagttttwtttttggtccagagtacttgttttcccgatgtgtccagccagctccgcagcagcttttca

SEQ ID NO: 15 - DNA, 121 bp, Homo sapiens
tatataaatatattaaaaaggaaaatgtttcagatgtttatttgtataattacttgattcmcacagtgagaaaaaatgaatgtattcctgtttttgaagagaagaataattttttttttct

SEQ ID NO: 16 - DNA, 121 bp, Homo sapiens
tatttgtataattacttgattcacacagtgagaaaaaatgaatgtattcctgtttttgaaragaagaataattttttttttctctagggagaggtacagtgtttatattttggagccttcc

SEQ ID NO: 17 - DNA, 121 bp, Homo sapiens
gaaggtgtaaaattgtaaatatttttatctatgagtaaatgttaagtagttgttttaaaawacttaataaaataattcttttcctgtggaagagaaagatggtctcctgagttttttaaaa

SEQ ID NO: 18 - protein, 412 aa, Homo sapiens
MERIPSAQPPPACLPKAPGLEHGDLPGMYPAHMYQVYKSRRGIKRSEDSKETYKLPHRLIEKKRRDRINECIAQLKDLLPEHLKLTTLGHLEKAVVLELTLKHVKALTNLIDQQQQKIIALQSGLQAGELSGRNVETGQEMFCSGFQTCAREVLQYLAKHENTRDLKSSQLVTHLHRVVSELLQGGTSRKPSDPAPKVMDFKEKPSSPAKGSEGPGKNCVPVIQRTFAHSSGEQSGSDTDTDSGYGGESEKGDLRSEQPCFKSDHGRRFTMGERIGAIKQESEEPPTKKNRMQLSDDEGHFTSSDLISSPFLGPHPHQPPFCLPFYLIPPSATAYLPMLEKCWYPTSVPVLYPGLNASAAALSSFMNPDKISAPLLMPQRLPSPLPAHPSVDSSVLLQALKPIPPLNLETKD

SEQ ID NO: 19 - DNA, 2922 bp, Homo sapiens
ggacaccgggccatgcacgcccccaactgaagctgcatctcaaagccgaagattccagcagcccaggggatttcaaagagctcagactcagaggaacatctgcggagagacccccgaagccctctccagggcagtcctcatccagacgctccgctagtgcagacaggagcgcgcagtggccccggctcgccgcgccatggagcggatccccagcgcgcaaccaccccccgcctgcctgcccaaagcaccgggactggagcacggagacctaccagggatgtaccctgcccacatgtaccaagtgtacaagtcaagacggggaataaagcggagcgaggacagcaaggagacctacaaattgccgcaccggctcatcgagaaaaagagacgtgaccggattaacgagtgcatcgcccagctgaaggatctcctacccgaacatctcaaacttacaactttgggtcacttggaaaaagcagtggttcttgaacttaccttgaagcatgtgaaagcactaacaaacctaattgatcagcagcagcagaaaatcattgccctgcagagtggtttacaagctggtgagctgtcagggagaaatgtcgaaacaggtcaagagatgttctgctcaggtttccagacatgtgcccgggaggtgcttcagtatctggccaagcacgagaacactcgggacctgaagtcttcgcagcttgtcacccacctccaccgggtggtctcggagctgctgcagggtggtacctccaggaagccatcagacccagctcccaaagtgatggacttcaaggaaaaacccagctctccggccaaaggttcggaaggtcctgggaaaaactgcgtgccagtcatccagcggactttcgctcactcgagtggggagcagagcggcagcgacacggacacagacagtggctatggaggagaatcggagaagggcgacttgcgcagtgagcagccgtgcttcaaaagtgaccacggacgcaggttcacgatgggagaaaggatcggcgcaattaagcaagagtccgaagaaccccccacaaaaaagaaccggatgcagctttcggatgatgaaggccatttcactagcagtgacctgatcagctccccgttcctgggcccacacccacaccagcctcctttctgcctgcccttctacctgatcccaccttcagcgactgcctacctgcccatgctggagaagtgctggtatcccacctcagtgccagtgctatacccaggcctcaacgcctctgccgcagccctctctagcttcatgaacccagacaagatctcggctcccttgctcatgccccagagactcccttctcccttgccagctcatccgtccgtcgactcttctgtcttgctccaagctctgaagccaatcccccctttaaacttagaaaccaaagactaaactctctaggggatcctgctgctttgctttccttcctcgctacttcctaaaaagcaacaaaaaagtttttgtgaatgctgcaagattgttgcattgtgtatactgagataatctgaggcatggagagcagattcagggtgtgtgtgtgtgtgtgtgtgtgtgtgtgtatgtgcgtgtgcgtgcacatgtgtgcctgcgtgttggtataggactttaaagctccttttggcatagggaagtcacgaaggattgcttgacatcaggagacttgggggggattgtagcagacgtctgggcttttccccacccagagaatagcccccttcgatacacatcagctggattttcaaaagcttcaaagtcttggtctgtgagtcactcttcagtttgggagctgggtctgtggctttgatcagaaggtactttcaaaagagggctttccagggctcagctcccaaccagctgttaggaccccacccttttgcctttattgtcgacgtgactcaccagacgtcggggagagagagcagtcagaccgagctttctgctaacatggggaggtagcaggcactggcatagcacggtagtggtttggggaggtttccgcaggtctgctccccacccctgcctcggaagaataaagagaatgtagttccctactcaggctttcgtagtgattagcttactaaggaactgaaaatgggccccttgtacaagctgagctgccccggagggagggaggagttccctgggcttctggcacctgtttctaggcctaaccattagtacttactgtgcagggaaccaaaccaaggtctgagaaatgcggacaccccgagcgagcaccccaaagtgcacaaagctgagtaaaaagctgcccccttcaaacagaactagactcagttttcaattccatcctaaaactccttttaaccaagcttagcttctcaaaggcctaaccaagccttggcaccgccagatcctttctgtaggctaattcctcttgcccaacggcatatggagtgtccttattgctaaaaaggattccgtctccttcaaagaagttttatttttggtccagagtacttgttttcccgatgtgtccagccagctccgcagcagcttttcaagatgcactatgcctgattgctgatcgtgttttaactttttcttttcctgtttttattttggtattaagtcgttgcctttatttgtaaagctgttataaatatatattatataaatatattaaaaaggaaaatgtttcagatgtttatttgtataattacttgattcacacagtgagaaaaaatgaatgtattcctgtttttgaagagaagaataatttttttttctctagggagaggtacagtgtttatattttggagccttcctgaaggtgtaaaattgtaaatatttttatctatgagtaaatgttaagtagttgttttaaaatacttaataaaataattcttttcctgtggaag

SEQ ID NO: 20 - DNA, 9372 bp, Homo sapiens
tagatagtaacagtatagactcattcagcacacagtgtaaggccaccaagatgctgagtcccaataggaaagaagagaaagatcccatctttctcttgtacgaaattatgttctctccccacttagaagagacaaaacagcagggacggctacaaaatggcaggattacccagtgcagctgctcagtgccacattgcacaactccagagggtgctctcacacagactgcaatgtgaatagctccccctggagttgagcggtgcacaacccaaacaaccctaccaggcaaccgtaagtatagtgattaagagcacaagctttggagtccaacaaacctgggggcctgtctcagactgaatcccagactatataacctggggcaggttatttagcctccttgagcctcaattttccaaacagaatatgttatacatccctagggcctatttcagtgtctggcattgaataacaataaacacgtgtcaaatgcatcgagctaatactagtacctgcctaaaaggtggtggtgaggagcaaagaaattgacatacgaaaggtttttaatatctacaaaagatgacagactgaaatagtaacattctccaggatggaaagatctatgccacgtaggaattgtttgttcaaattattattttttaaaagccataagctatggagagaataacagagttttgttttgttttttaatcctgatacggtggaagtctggtaggatgggaaccgttcttgaagcttgagaatacattcggaacaaataaaaagtaggaaatgcatacattttgttatttacagttgtagggcaggtaagggcaaaacgtattgataaactcaaagaattttaagaatgatattgtcatgagccatgagccatggaagctgcaaggtttgggctccctcctgccatcgggtggggggtgtcggcggaagggggtacgtgtcctgtcctctcccagtgcttatttcctgctggaaccgtggttttgacgcagccgtgacagcccttcatccttattaaaatcacacaatgaagcaggtcgcccaagattgaaagcgcgtttccagggcggaccccggtccccgcctttggccagcaaaagtacattctggcagggagggatctctgagccccgaagtgaggtcatgccagttagcagttagaaaaggaggcatttctcacacttgctaaagagtttgacgcttgtgaaatataaacaggaagcgggagctctccaaggcgacctgaaggaaatacactgccagtttggtcaacttgtgaatgccagttctctaggtcgtcaagggcactgccagttaagactgatcccctaactgcacccgaggctactactcttcgcccagtctcggcagtgaccccatttttttttcaatccttcccacagccaggtcactcagcagtggggtgactgacccgaagcgcagtgggaagggcttacagtcatctctcaactccccgaacttgcagggcaggggagcgcagaggtcagtgaaatccccagccaggccttattttagtcatgagtttccactggctccctcctctgactgtatcagcgctgctcacaccgcgggattggagtttcagctgttttccctgctcccggctgagtgagaaggtcacagagcccggacctttccgggggaggtgggtggggccctccggcagcctaagtgagattgcagagatctggggccgctggaggaggtccttccgccccgggcttggggctttgggttagcttctcccccgcttctctacctaggcatctcggttacaatgaaattaaaaacaaaacacaaagcaacacaagttctcattgtttttcccctctgtcagccagtggcctcgatacaattttcccagggctgcctttcctttattgctcaatttaacctcttcctagaggttctgcaagttcaagggcaggatccgtgtcggattcatcaactagtgcagcgcctagcgcagagcagggggctcaagaaatacgtgtcgaccgcatgattgctaagatttccctgtaacaggcggtttttttttcttgtctctctcatccctcctgccctgggtgcagaccgcgaacagcgccgccgaacttcccaacaggcagggagagggccgatccgggctggcacgccacgtccccggggtctcagtccctgggtcaacgcagagaggctcgcggtccgcggtgctacgacctgagcccggccacgtgaagcgtcacgccctgcgctgctgcagggcaggcgccgccgccgcggttgtgtaacgctgcagccggaggggaggcgaccggggcgcctctgctccggagggggcagaggagggagggagcgccgggcacagcgcccggtatctttctgcgcttgactggccgggaggaagggggtgacactggggcacccagggggctgcgcaaccgggtgcccgcccgcccgcaggttgtgcccaggagcggagctcctgggagcgaggggccctcggctcacaccctccacggtcaggtgcgcgcggcgtgctcggcggcagcctcagctgcccctgcgcttggccgtcggcccgcttcccatggggtgacatccgccccgcccctcggtccctccccaaggcgggcaattcctggacgcgagggtgagcagtgggggcagggaagccaggacggaaagaaaccccagcctctggggaaggctggggggccgggcgactccctccgcagcgcgccggttcctccaagcgggccgggcgggggaggaggaagagggctgggctggagctagcaaggggatattcctctcccggcttgagtcagacgcgggcggatccgtcctcccccgttccctcccaggagacgggaacttacttcatttccctggggcaggttcgcccacgttaccaacttctccccctcccccagcacccccgtcccttccagcttccgcgccccccacccaactgggcaggacccaggtcgtgctgccaccccctcttcggggaaaggcggccgcagccgcagacacctgggggccggggctgggggtgggggctccctagcagccgccggagcgttgtccaacacgtgagactcatgtgatgaagccgggggagggcgggcaggtcgctccttccctccccggcagtggccagacgtgcctggagtcacagggtagaacacgtagctccaacccacccactcgctcccatttaacccagcccgcagcctctccattactcctcggcttgcccccccacccccccactccgccctaaccgcccccccctcacccgctccctcccgcctccccgcccgccccacttctcattcacttggctcgcacggcgcagacagaccgcgcagggagcacacaccgccagtctgtgcgctgagtcggagccagaggccgcggggacaccgggccatgcacgcccccaactgaagctgcatctcaaagccgaagattccagcagcccaggggatttcaaagagctcagactcagaggaacatctgcggagagacccccgaagccctctccagggcagtcctcatccagacgctccgctagtgcagacaggagcgcgcagtggccccggctcgccgcgccatggagcggatccccagcgcgcaaccaccccccgcctgcctgcccaaagcaccgggactggagcacggagacctaccagggtaagttggcactccttggcccttcaagcctcaactgcagccccatgttatgcgccactctcaacttggagcagctccgggcgcaccgggaggttcttgcccggcagaacgcctgcagccgtgcgcaagcagtcacgacccttcctggtctctcttcctgcaggatgtaccctgcccacatgtaccaagtgtacaagtcaagacggggaataaagcggagcgaggacagcaaggtaagcaagtgcacccctagggaccctgcgctcagcccctcgcgcgcgctgagtttccaagaaaagttttctcgctttgaggttggcgaggggatgcagggtgtgggctcggtgcctcttctgagtgacttggaaaagaaagggcagacgatggttctgggtgctactctgctactctgctcctctgccggactgttgctgcggggctgggaggataggcttctcatctccttccccaagcgcccaccgcctccccgtgcgtcttgcaggagacctacaaattgccgcaccggctcatcgagaaaaagagacgtgaccggattaacgagtgcatcgcccagctgaaggatctcctacccgaacatctcaaacttacagtaagtgagaagctggcccctttccaacccagtcctttgcccagagggcgggcgggtcactcgccgcctgcaggttccgcggggaactgcagaccagactggcggatcagagctggcgggtggctctgcggtggggtcctcctccacagtcctggcgttttctggctgactcatgcaaatgaggcgagaaccacttggacctctccaagttggctccaacaccaactggccttccttttcaggagtttaacttctggtgattttcaggattccctagtttttctcacttaaaggaacacgccaagaaacacacacattttatttaccccttcccctctaccactccccccaccttttttgttgttgttgtcaaataaggggacttgctttggtgtgtgatttttacggcctgtcctttcttgacagtagccctcggctacttcgcagcctttggttggggaaatggaaaaaaaaaaaatagtatttcgggaggaacaaaaaagcctggatgagctggaagttctgaaagggtgtaaggaaagaagtagggaaagcctcttggctgccagacgtaatagaggtccctctctgagttgacaccactgcaggaaaatgttatctctggattcccctgagttaaggtcggcagaggctgctggagaaatttatctgtcaaaggattttcctgaggtagttgcagcttccaaaatgcctccaaagacaggtggcacccgtttttttaaattatttaattatatatatatattatatatatatatacacacacacacacacacacatatatgagtgtatctgtctatctttttaaaagcatgacctacagacttaaaacagccaaccaggaaacactattccacttttttttttcttttctcatttctcattgctaataaatgtcccaaaggtgggacttctctgacctcaccgctgactaaggctcttttccttccagactttgggtcacttggaaaaagcagtggttcttgaacttaccttgaagcatgtgaaagcactaacaaacctaattgatcagcagcagcagaaaatcattgccctgcagagtggtttacaagctggtgagtgctgattctggctatgctctctttaagagtttgagtgcaaatagagagcccgtgggctcaacatctgatgtaataaacattactgcaacaaattgcttatgcacgttcattgggggagatgcagttttatctttcctaaaaggtcagacttttcatttgaaatgcattgtgccacctggcatgaatacgtattagtgaaatgataagatattcctgtggcattttagcttgagagtctctgggttctgggagatttccctactcccttttaaaatgccaaggcacctataatttgcattgtgggtgttttacagttggtcttcttagccctctttcattggagaagaaatcatgggtatgccccagcgttttcctgtttctttgcttccttgtcacttggtgtgtggtgtgatcctggcaggatacagcaggagtttttcctgcatttcatgtggttccctgaatagccactaggtatcttggtgaattgcacaatctagcgaaaccactgaagtttcatcattcgtttctactgacttccagataatcggagtcaaccttctaaccttctagtctcacttcttccaaataatactgtacagactggggagaattattctacccactccctcatttcatgcttgtctgccttcctctcgaaggctcgtatgatgaaaattgcaaaaacccagctagtgcttctgcctgtttcctcattcaaaataggaagatggtcatacctaatgtcttgaggatcatggatagaatatgtggagaagacatagaaatagcctaattaaaggtcacagcatgactgcaacttttacaaatcattgaaaataagtttactttaggtgattgttgtttcttcattcttttcctcattttataactatccagcctctgcccattttgccacggtgcaatgttgtatgcattgactcttaggtttataaatacatcagaatgttctttgctgcctcgcttacatggaaaactcacaaaccacctatacaatccaaaagaggggaaacagctcatctcatattaattatggtccatttccatgataggatattatgcaaccattaaaaatcatattttatctgagtgttagtatatggattattttaattatttatacttttcagtgtaattcttctctgagcatactacttttggagttaggaacacaaatcagtaaatgcattttttttttttaacaaaaactaaagcacctggttttttggagtatagtttccaggtgcgccacaggccttctgaaacgttttccttcggatttttctttccccaggtgagctgtcagggagaaatgtcgaaacaggtcaagagatgttctgctcaggtttccagacatgtgcccgggaggtgcttcagtatctggccaagcacgagaacactcgggacctgaagtcttcgcagcttgtcacccacctccaccgggtggtctcggagctgctgcagggtggtacctccaggaagccatcagacccagctcccaaagtgatggacttcaaggaaaaacccagctctccggccaaaggttcggaaggtcctgggaaaaactgcgtgccagtcatccagcggactttcgctcactcgagtggggagcagagcggcagcgacacggacacagacagtggctatggaggagaatcggagaagggcgacttgcgcagtgagcagccgtgcttcaaaagtgaccacggacgcaggttcacgatgggagaaaggatcggcgcaattaagcaagagtccgaagaaccccccacaaaaaagaaccggatgcagctttcggatgatgaaggccatttcactagcagtgacctgatcagctccccgttcctgggcccacacccacaccagcctcctttctgcctgcccttctacctgatcccaccttcagcgactgcctacctgcccatgctggagaagtgctggtatcccacctcagtgccagtgctatacccaggcctcaacgcctctgccgcagccctctctagcttcatgaacccagacaagatctcggctcccttgctcatgccccagagactcccttctcccttgccagctcatccgtccgtcgactcttctgtcttgctccaagctctgaagccaatcccccctttaaacttagaaaccaaagactaaactctctaggggatcctgctgctttgctttccttcctcgctacttcctaaaaagcaacaaaaaagtttttgtgaatgctgcaagattgttgcattgtgtatactgagataatctgaggcatggagagcagattcagggtgtgtgtgtgtgtgtgtgtgtgtgtgtgtatgtgcgtgtgcgtgcacatgtgtgcctgcgtgttggtataggactttaaagctccttttggcatagggaagtcacgaaggattgcttgacatcaggagacttgggggggattgtagcagacgtctgggcttttccccacccagagaatagcccccttcgatacacatcagctggattttcaaaagcttcaaagtcttggtctgtgagtcactcttcagtttgggagctgggtctgtggctttgatcagaaggtactttcaaaagagggctttccagggctcagctcccaaccagctgttaggaccccacccttttgcctttattgtcgacgtgactcaccagacgtcggggagagagagcagtcagaccgagctttctgctaacatggggaggtagcaggcactggcatagcacggtagtggtttggggaggtttccgcaggtctgctccccacccctgcctcggaagaataaagagaatgtagttccctactcaggctttcgtagtgattagcttactaaggaactgaaaatgggccccttgtacaagctgagctgccccggagggagggaggagttccctgggcttctggcacctgtttctaggcctaaccattagtacttactgtgcagggaaccaaaccaaggtctgagaaatgcggacaccccgagcgagcaccccaaagtgcacaaagctgagtaaaaagctgcccccttcaaacagaactagactcagttttcaattccatcctaaaactccttttaaccaagcttagcttctcaaaggcctaaccaagccttggcaccgccagatcctttctgtaggctaattcctcttgcccaacggcatatggagtgtccttattgctaaaaaggattccgtctccttcaaagaagttttatttttggtccagagtacttgttttcccgatgtgtccagccagctccgcagcagcttttcaaratgcactatgcctgattgctgatcgtgttttaactttttcttttcctgtttttattttggtattaagtcgttgcctttatttgtaaagctgttataaatatatattatataaatatattaaaaaggaaaatgtttcagatgtttatttgtataattacttgattcacacagtgagaaaaaatgaatgtattcctgtttttgaagagaagaataattttttttttctctagggagaggtacagtgtttatattttggagccttcctgaaggtgtaaaattgtaaatatttttatctatgagtaaatgttaagtagttgttttaaaatacttaataaaataattcttttcctgtggaagagaaagatggtctcctgagttttttaaaatagctcacttggtgtgatatggctgactcccatgtccttctttcttggctgaagcaaaccagttaatctcagtgtaatttcattcagcttttcatgtgctttgtattcaatcagaataagcttagtcaacctggcaggtaaatattggttcaatttttgtttaacccttgccaggggttcatgatcatcctctacctctttccagagttaagtaaaaagcatattttcccttgccctgctttcatcg

SEQ ID NO: 21 - DNA, 60 bp, Homo sapiens
ccgcgaacagcgccgccgaacttcccaacaggcagggagagggccgatccgggctggcac

SEQ ID NO: 22 - DNA, 60 bp, Homo sapiens
ggaggcgaccggggcgcctctgctccggagggggcagaggagggagggagcgccgggcac

SEQ ID NO: 23 - DNA, 60 bp, Homo sapiens
cctcatccagacgctccgctagtgcagacaggagcgcgcagtggccccggctcgccgcgc

SEQ ID NO: 24 - DNA, 60 bp, Homo sapiens
caggatgtaccctgcccacatgtaccaagtgtacaagtcaagacggggaataaagcggag

SEQ ID NO: 25 - DNA, 60 bp, Homo sapiens
agacaggtggcacccgtttttttaaattatttaattatatatatatattatatatatata

SEQ ID NO: 26 - DNA, 60 bp, Homo sapiens
aatgcctccaaagacaggtggcacccgtttttttaaattatttaattatatatatatatt

SEQ ID NO: 27 - DNA, 60 bp, Homo sapiens
atcagacccagctcccaaagtgatggacttcaaggaaaaacccagctctccggccaaagg

SEQ ID NO: 28 - DNA, 60 bp, Homo sapiens
actgcgtgccagtcatccagcggactttcgctcactcgagtggggagcagagcggcagcg

SEQ ID NO: 29 - DNA, 60 bp, Homo sapiens
ctgcaagattgttgcattgtgtatactgagataatctgaggcatggagagcagattcagg

SEQ ID NO: 30 - DNA, 60 bp, Homo sapiens
ctttaaagctccttttggcatagggaagtcacgaaggattgcttgacatcaggagacttg

SEQ ID NO: 31 - DNA, 60 bp, Homo sapiens
aagctccttttggcatagggaagtcacgaaggattgcttgacatcaggagacttgggggg

SEQ ID NO: 32 - DNA, 60 bp, Homo sapiens
tccttttggcatagggaagtcacgaaggattgcttgacatcaggagacttgggggggatt

SEQ ID NO: 33 - DNA, 60 bp, Homo sapiens; identical to SEQ ID NO: 32

SEQ ID NO: 34 - DNA, 60 bp, Homo sapiens
caacggcatatggagtgtccttattgctaaaaaggattccgtctccttcaaagaagtttt

SEQ ID NO: 35 - DNA, 60 bp, Homo sapiens
tatataaatatattaaaaaggaaaatgtttcagatgtttatttgtataattacttgattc

SEQ ID NO: 36 - DNA, 60 bp, Homo sapiens
tatttgtataattacttgattcacacagtgagaaaaaatgaatgtattcctgtttttgaa

SEQ ID NO: 37 - DNA, 60 bp, Homo sapiens
gaaggtgtaaaattgtaaatatttttatctatgagtaaatgttaagtagttgttttaaaa

SEQ ID NO: 38 - DNA, 60 bp, Homo sapiens
ccacgtccccggggtctcagtccctgggtcaacgcagagaggctcgcggtccgcggtgct

SEQ ID NO: 39 - DNA, 60 bp, Homo sapiens
gcgcccggtatctttctgcgcttgactggccgggaggaagggggtgacactggggcaccc

SEQ ID NO: 40 - DNA, 60 bp, Homo sapiens
atggagcggatccccagcgcgcaaccaccccccgcctgcctgcccaaagcaccgggactg

SEQ ID NO: 41 - DNA, 60 bp, Homo sapiens
gaggacagcaaggtaagcaagtgcacccctagggaccctgcgctcagcccctcgcgcgcg

SEQ ID NO: 42 - DNA, 60 bp, Homo sapiens
acacacacacacacacacacatatatgagtgtatctgtctatctttttaaaagcatgacc

SEQ ID NO: 43 - DNA, 41 bp, Homo sapiens
atatatgagtgtatctgtctatctttttaaaagcatcacct

SEQ ID NO: 44 - DNA, 60 bp, Homo sapiens
tcggaaggtcctgggaaaaactgcgtgccagtcatccagcggactttcgctcactcgagt

SEQ ID NO: 45 - DNA, 60 bp, Homo sapiens
cacggacacagacagtggctatggaggagaatcggagaagggcgacttgcgcagtgagca

SEQ ID NO: 46 - DNA, 60 bp, Homo sapiens
atgtgcgtgtgcgtgcacatgtgtgcctgcgtgttggtataggactttaaagctcctttt

SEQ ID NO: 47 - DNA, 60 bp, Homo sapiens
gggggattgtagcagacgtctgggcttttccccacccagagaatagcccccttcgataca

SEQ ID NO: 48 - DNA, 60 bp, Homo sapiens
attgtagcagacgtctgggcttttccccacccagagaatagcccccttcgatacacatca

SEQ ID NO: 49 - DNA, 60 bp, Homo sapiens
tagcagacgtctgggcttttccccacccagagaatagcccccttcgatacacatcagctg

SEQ ID NO: 50 - DNA, 60 bp, Homo sapiens; identical to SEQ ID NO: 49

SEQ ID NO: 51 - DNA, 60 bp, Homo sapiens
tttttggtccagagtacttgttttcccgatgtgtccagccagctccgcagcagcttttca

SEQ ID NO: 52 - DNA, 60 bp, Homo sapiens
cacagtgagaaaaaatgaatgtattcctgtttttgaagagaagaataattttttttttct

SEQ ID NO: 53 - DNA, 60 bp, Homo sapiens
agaagaataattttttttttctctagggagaggtacagtgtttatattttggagccttcc

SEQ ID NO: 54 - DNA, 60 bp, Homo sapiens
acttaataaaataattcttttcctgtggaagagaaagatggtctcctgagttttttaaaa

(19) United States
(12) Patent Application Publication
(10) Pub. No.: US 2006/0166217 A1
(43) Pub. Date: Jul. 27, 2006

(54) Genetic marker for coronary artery disease

(75) Inventors: Bjorn Carlsson, Molndal (SE); Per Georg Olsson, Molndal (SE)

Correspondence Address: FISH & NEAVE IP GROUP;ROPES & GRAY LLP, ONE INTERNATIONAL PLACE, BOSTON, MA, 02110-2624, US

(73) Assignee: AstraZeneca AB, Sodertalje (SE)

(21) Appl. No.: 10/565,263

(22) PCT Filed: Jul. 21, 2004
(86) PCT No.: PCT/GB04/03153

§ 371 Date: Jan. 18, 2006

(30) Foreign Application Priority Data
Jul. 22, 2003 (SE) 0302122-7

(51) Int. Cl. C12Q 1/68; A61K 48/00; A61K 39/395

(52) U.S. Cl. 435/6; 514/44; 424/143.1

(57) ABSTRACT

The present invention describes a Coronary artery disease (CAD) susceptibility gene and the use of this gene in the development of novel therapies to treat CAD and in the diagnosis of susceptibility to CAD.

### FIELD OF THE INVENTION

The present invention describes a coronary artery disease susceptibility gene and its use in the diagnosis and therapy of CAD.

### BACKGROUND

Coronary artery disease (CAD) is the most common type of heart disease. It is a consequence of atherosclerosis, which is a progressive inflammatory tissue response resulting from deposition of modified lipoproteins in the arterial intima of the vascular wall. This process can lead to development of complex atherosclerotic lesions or plaques at pre-existing focal arterial intima cushions rich in extracellular matrix (Glass et al, Cell 104, 503-516, 2001). Overtime these lesions narrow the coronary arteries, restricting blood flow to the heart and can cause angina. Complete blockage can lead to myocardial infarction.

The known risk factors for CAD include type II diabetes, insulin resistance, obesity (Rao et al, Am. Heart J. 142, 1102-1107, 2001), hyperlipidemia, high blood pressure, cigarette smoking and physical inactivity. CAD is also known to have a significant genetic component.

There are no drugs currently available to prevent CAD. Efforts have been focussed at measures to slow down CAD, and different drugs and surgical techniques are available to repair clogged coronary arteries. However, there is a need for a better understanding of the pathophysiology of CAD and the development of specific and effective drugs.

To develop more effective drugs, it is critical that the susceptibility genes for CAD are identified. As mentioned above, the pathogenesis of CAD has been shown by twin and other studies to include a significant genetic component, and the effects of genes on susceptibility to CAD are likely to be particularly strong at younger ages (Marenberg et al, New England J Med 330, 1041-1046, 1994). However, CAD is unlikely to be inherited as a simple Mendelian trait, but is instead a complex multifactorial disorder in which the phenotype is heavily influenced by environmental as well as genetic factors. Although a number of loci have been identified for risk factors related to CAD, only a limited number of genome scanning studies have been published showing linkage to CAD. For example, CAD has been linked to chromosome 2 and X (Pajukanta et al, Am. J. Hum. Genet. 67, 1481-1493, 2000), chromosome 16 (Francke et al., Human Molec. Genet. 10, 2751-2765, 2001) and chromosome 14 (Broeckel et al., Nature Genetics 30, 210, 2002).

### SUMMARY OF THE INVENTION

The present invention describes the identification of a CAD susceptibility gene, a basic helix-loop-helix transcription factor 2 (BHLHB2). The identification of BHLHB2 as a CAD susceptibility gene enables the development of novel therapies for CAD by screening for compounds and other entities, such as antibodies, which modulate the activity of BHLHB2. Knowledge of the BHLHB2 gene sequence also enables the development of novel antigene methods to modulate the expression of the associated gene and may also enable the development of novel gene therapy techniques to treat CAD. The discovery of the linkage between BHLHB2 and CAD may also assist in developing novel methods for diagnosing CAD via (i) measuring the levels of the translated mRNA of BHLHB2 present in affected tissue and (ii) measuring the levels of the BHLHB2 protein in affected tissue. It is possible that the diagnosis of CAD, or the susceptibility of an individual to CAD, by these methods may be achieved in patients who do not yet display the classical symptoms of the disease. Such determination of susceptibility to CAD, or the early detection of disease development, will lead to earlier clinical intervention than is currently possible and will lead to more effective treatment of the disease.

Specifically, the invention includes a method of identifying a test compound that modulates the expression of a BHLHB2 gene, includes contacting a cell capable of expressing a BHLHB2 gene with a test compound; and determining the level of expression of the BHLHB2 gene in the presence of the test compound, wherein a decrease or an increase in BHLHB2 gene expression, as compared to the level of expression of BHLHB2 in the absence of the compound, is indicative that the test compound modulates BHLHB2 gene expression.

Also within the invention is a method of identifying a test compound that modulates the activity of a protein encoded by the BHLHB2 gene, including contacting the protein with a test compound and determining the level of activity of the BHLHB2 protein in the presence of the compound, wherein a decrease or an increase in the protein activity, as compared to the level of activity of the BHLHB2 protein in the absence of the compound, is indicative that the test compound modulates BHLHB2 protein activity.

The invention further includes a method for determining if a BHLBH2 gene has an altered level of gene expression in a CAD cell. The method includes comparing the level of BHLBH2 gene expression in a cell from a patient having CAD with a control cell (a cell from a patient not having CAD), and determining the level of expression of the BHLBH2 gene in both cells, wherein a decrease or an increase in expression of the BHLBH2 gene, as compared to the level of expression of the BHLBH2 gene in the control cell, indicates that the BHLBH2 has altered gene expression.

Also within the invention is a method for determining the level of a BHLBH2 protein in a CAD patient compared to a control including comparing the protein level of BHLBH2 in a cell from a patient having CAD with a control cell (a cell from a patient not having CAD), and determining the level of the BHLBH2 protein in both cells.

In another aspect, the invention includes a method of identifying a binding partner of the BHLHB2 protein including contacting a BHLHB2 protein with a test target protein, and determining if the test target protein can interact with the BHLHB2 protein, wherein interaction of the test target protein with BHLBH2 indicates that the test target protein is a BHLBH2 binding partner. In one embodiment, the method can further include contacting a gene encoding the test target protein with a test compound; and determining the level of expression of the test target gene in the presence of the test compound, wherein a decrease or an increase in test target gene expression, as compared to the level of expression of the test target gene in the absence of the compound, is indicative that the test compound modulates expression of the test target gene and is useful in the treatment of CAD. In another embodiment, the method further includes contacting the test target protein with a test compound; and determining the level of activity of the test target protein in the presence of the test compound, wherein a decrease or an increase in test target protein activity, as compared to the level of activity of the test target protein in the absence of the compound, is indicative that the test compound modulates test target protein activity and is useful in the treatment of CAD.

The invention further features a method of treating a subject having CAD comprising administering an effective amount of the compound identified above. The invention further includes a pharmaceutical composition comprising the compound identified as above.

In another aspect, the invention includes a method of identifying other components of the CAD biochemical pathway of which BHLHB2 is a component.

The invention further includes methods of diagnosing CAD or a susceptibility thereto in a subject. The method includes determining the level of a BHLHB2 protein in a sample from a subject; and comparing the level of the protein in the sample with a control, wherein a decrease or an increase in the level of the protein in the sample compared to the control indicates that the subject has CAD, or a susceptibility thereto. The invention also extends to products useful for carrying out the assay, such as DNA probes (labelled or unlabelled), kits and the like.

The present invention also includes gene-based therapies directed at the BHLHB2 gene. Therapies may be in the form of polynucleotides comprising all or a portion of the BHLHB2 gene, placed in appropriate vectors or delivered to target cells in direct ways which would modify the function of the BHLHB2 protein.

As used herein, a “CAD susceptibility gene” refers to a gene that has a predisposing influence on the development of CAD in a subject.

As used herein, “coronary artery disease” (CAD) refers to disorders and conditions related to the deposition of atheroma in the large- and medium-sized arteries serving the heart. Coronary artery disease means clinical syndromes (including, but not limited to, angina, myocardial infarction, unstable angina, and sudden ischemic death) which are based on the pathology of coronary artery atheroma.

As used herein “BHLHB2 protein” refers to the BHLHB2 protein, peptide fragments thereof, mutants, variants, truncated forms of BHLHB2, and fusion proteins of BHLHB2.

### BRIEF DESCRIPTION OF THE DRAWINGS

FIG. 1 is a line graph showing the KAC LOD-score of chromosome 3.

### DETAILED DESCRIPTION OF THE INVENTION

The present invention describes a CAD susceptibility gene, BHLHB2. BHLHB2, also known as DEC1, is a basic helix-loop-helix transcription factor and has been implicated in control of chondrocyte (Shen-M et al. BBRC, 236;294-289, 1997), neuronal (Boudjelal, Genes and Dev 11;2052-2065, 1997) and preadipocyte differentiation (Inuzuka-H et al. BBRC 265:664-668, 1999).

The present finding shows that BHLHB2, which is mapped to chromosome region 3p, shows linkage with DNA markers in CAD affected individuals. The BHLHB2 gene and amino acid sequences are known for many different species, e.g., the human BHLHB2 nucleic and amino acid sequences are available on genbank (http://www.ncbi.nlm.nih.gov/), under accession number AB043885 (formerly XM 005086), and the mouse BHLHB2 is available on genbank under accession number NM 011498.

Knowledge of the BHLHB2 sequence enables the development of novel antigene methods to modulate the expression of BHLHB2 and also enables the development of novel gene therapy techniques to treat CAD.

The sequence of human BHLHB2 protein, as disclosed in NM003670 and AB043885, is depicted in SEQ ID NO: 18, the cDNA sequence in SEQ ID NO: 19 and the genomic sequence in SEQ ID NO:20.

Functional Assay

To further evaluate the role of BHLHB2 in CAD, various functional assays can be performed. For example techniques such as Northern analysis, in situ hybridization or expression profiling on cDNA microarrays can be used to further verify the association of BHLHB2 with CAD. In one example, a reporter-based assay may be devised to detect whether the BHLHB2 gene has a different transcription level and/or message stability compared to the same gene in a person not susceptibile to CAD. Individuals who carry the BHLHB2 gene may exhibit differences in their ability to regulate protein biosynthesis under different physiological conditions and may display altered physiological abilities.

In another example, the level of BHLHB2 gene expression can be assayed by detecting and measuring BHLHB2 transcription. For example, RNA from a cell type or tissue known, or suspected of having CAD, can be isolated and tested utilizing hybridization or PCR techniques such as those described above. The isolated cells can be obtained from cell culture or from a patient. The analysis of cells taken from culture may be a necessary step in the assessment of cells to be used as part of a cell-based gene therapy technique or, alternatively, to test the effect of compounds on the expression of BHLHB2. Such analyses can reveal both quantitative and qualitative aspects of the expression pattern of the BHLHB2 gene, including activation or inactivation of BHLHB2 gene expression.

In one embodiment of such a detection scheme, cDNAs are synthesized from the RNAs of interest (e.g., by reverse transcription of the RNA molecule into cDNA). A sequence within the cDNA is then used as the template for a nucleic acid amplification reaction, such as a PCR amplification reaction, or the like. For detection of the amplified product, the nucleic acid amplification may be performed using radioactively or non-radioactively labeled nucleotides. Alternatively, enough amplified product can be made such that the product can be visualized by standard ethidium bromide staining or by utilizing any other suitable nucleic acid staining method.

Additionally, it is possible to perform BHLHB2 gene expression assays in situ, i.e., directly upon tissue sections (fixed and/or frozen) of patient tissue obtained from biopsies or resections, such that no nucleic acid purification is necessary (See, e.g., Nuovo, G. J., PCR In Situ Hybridization: Protocols And Applications, Raven Press, NY, 1992).

Alternatively, if a sufficient quantity of the appropriate cells can be obtained, standard Northern analysis can be performed to determine the level of mRNA expression of the BHLHB2 gene.

Also within the invention is the determination of whether BHLHB2 gene has a gene mutation compared to the wild-type gene. Alternative methods for the detection of BHLHB2 gene mutations in patient samples or other appropriate cell sources, may involve their amplification, for example, by PCR (the experimental embodiment set forth in U.S. Pat. No. 4,683,202), followed by the detection of the amplified molecules using techniques well known to those of skill in the art. The resulting amplified sequences can be compared to those which would be expected if the nucleic acid being amplified contained only normal copies of the BHLHB2 gene in order to determine whether a BHLHB2 gene mutation exists.

Additionally, well-known genotyping techniques can be performed to identify individuals carrying BHLHB2 gene mutations. Such techniques include, for example, TaqMan 5′ nuclease assay, allele specific PCR, primer extension, mass spectrometry and Pyrosequencing (reviewed in Kwok, Pharmacogenomics 1: 95-100, 2000)

Pathway Mapping

Also within the invention is the identification and elucidation of the CAD biochemical\signal transduction pathway of which BHLHB2 is a component. In particular, the invention includes identifying the other components of the BHLHB2 CAD biochemical pathway. In this way it is possible to identify the specific critical signaling pathway which links the disease stimulus to the cell's response and enables the identification of new potential targets for therapy intervention.

As used herein, a BHLHB2 “target molecule” is a molecule in the CAD biochemical pathway with which BHLHB2 binds or interacts, directly or indirectly, with, or is a molecule that regulates the expression of the CAD gene or translation of the protein. For example, the target molecule can be a protein which directly interacts with BHLHB2, or can be a protein which does not itself directly interact with the BHLHB2, but which is a component of the BHLHB2 CAD biochemical pathway. In one example, the target molecule can be a cell membrane or a cytoplasmic molecule. In another example, the target molecule can be an intercellular protein or a protein which facilitates the association of downstream or upstream signaling molecules with the BHLHB2 protein.

According to a further aspect of the invention there is provided the use of the BHLHB2 protein in research to identify further gene targets implicated in CAD. Methods for identifying proteins which interact with BHLHB2 are known in the art, e.g., the two-hybrid assay (see, e.g., U.S. Pat. No. 5,283,317; Zervos et al. Cell 72:223-232, 1993) or using cell culture techniques to identify binding partners.

The two-hybrid system is based on the modular nature of most transcription factors, which consist of separable DNA-binding and activation domains. Briefly, the assay utilizes two different DNA constructs. In one construct, the gene that codes for a BHLHB2 protein is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a BHLHB2-dependent complex, the DNA-binding and activation domains of the transcription factor are brought into close proximity. This proximity allows transcription of a reporter gene (e.g., LacZ) which is operably linked to a transcriptional regulatory site responsive to the transcription factor. Expression of the reporter gene can be detected and cell colonies containing the functional transcription factor can be isolated and used to obtain the cloned gene which encodes the “target” protein which interacts with the BHLHB2 protein.

Alternatively, binding partners for BHLHB2 can be identified using cell culture techniques or using cells obtained directly from a CAD patient. The method includes isolating the BHLHB2 protein from the cell and determining the identity of its target molecule. Initial screening can be accomplished by Western blot analysis to analyse immunochemically, e.g., using antibodies against the BHLBH2, the size of BHLBH2-target molecule complex. Further analysis of the complex will reveal the identity of the target molecule.

The gene and protein encoded by the target molecule is also a potential target for therapeutic intervention in CAD disease, for instance in the development of antisense nucleic acid targeted to the mRNA; or more widely in the identification or development of chemical or hormonal therapeutic agents. The person skilled in the art is also capable of devising screening assays to identify compounds (chemical or biological) that modulate (activate or inhibit) the identified gene, which compounds may prove useful as therapeutic agents in treating or preventing CAD.

BHLHB2 Proteins and Polypeptides

BHLHB2 proteins, peptide fragments, mutated, and truncated forms of the BHLHB2, and/or BHLHB2 fusion proteins, can be prepared for a variety of uses, including but not limited to the generation of antibodies, as reagents in diagnostic assays, in the identification of other cellular gene products involved in the pathogenesis of CAD, as reagents in assays for screening for compounds that can be used in the treatment of CAD, and as pharmaceutical reagents useful in the treatment of CAD.

A variety of host-expression vector systems may be utilized to express the BHLHB2 nucleotide sequences of the invention. The expression systems that may be used for purposes of the invention include but are not limited to microorganisms such as bacteria (e.g., E. coli, B. subtilis) transformed with recombinant bacteriophage DNA, plasmid DNA or cosmid DNA expression vectors containing BHLHB2 nucleotide sequences or mammalian cell systems (e.g., COS, CHO, BHK, 293, 3T3) harbouring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the BHLHB2 gene product being expressed. For example, when a large quantity of BHLHB2 is to be produced, for the generation of pharmaceutical compositions of BHLHB2 or for raising antibodies to the BHLHB2 protein, for example, vectors which direct the expression of high levels of fusion protein products that are readily purified may be desirable. Such vectors include, but are not limited to, the E. coli expression vector pUR278 (Ruther et al., EMBO J. 2:1791, 1983), in which the BHLHB2 coding sequence may be ligated individually into the vector in frame with the lacZ coding region so that a fusion protein is produced; pIN vectors (Inouye & Inouye, Nucleic Acids Res. 13:3101-3109, 1985; Van Heeke & Schuster, J. Biol. Chem. 264:5503-5509, 1989); and the like. PGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The PGEX vectors are designed to include thrombin or factor Xa protease cleavage sites so that the cloned target gene product can be released from the GST moiety.

In mammalian host cells, a number of viral-based expression systems may be utilized. In cases where an adenovirus is used as an expression vector, the BHLHB2 nucleotide sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the BHLHB2 in infected hosts. (E.g., See Logan & Shenk, , Proc. Natl. Acad. Sci. USA 81:3655-3659, 1984).

For long-term, high-yield production of recombinant proteins, stable expression is preferred. For example, cell lines which stably express the BHLHB2 sequence described above may be engineered. Rather than using expression vectors which contain viral origins of replication, host cells can be transformed with DNA controlled by appropriate expression control elements (e.g., promoter, enhancer sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the BHLHB2. Such engineered cell lines may be particularly useful in screening and evaluation of compounds that affect the endogenous activity of the BHLHB2.

Alternatively, any fusion protein may be readily purified by utilizing an antibody specific for the fusion protein being expressed. For example, a system described by Janknecht et al. allows for the ready purification of non-denatured fusion proteins expressed in human cell lines (Janknecht, et al., Proc. Natl. Acad. Sci. USA 88:8972-8976, 1991). In this system, the gene of interest is subcloned into a vaccinia recombination plasmid such that the gene's open reading frame is translationally fused to an amino-terminal tag consisting of six histidine residues. Extracts from cells infected with recombinant vaccinia virus are loaded onto Ni.2+. nitriloacetic acid-agarose columns and histidine-tagged proteins are selectively eluted with imidazole-containing buffers.

The BHLHB2 protein can also be expressed in transgenic animals. Animals of any species, including, but not limited to, mice, rats, rabbits, guinea pigs, pigs, micro-pigs, goats, and non-human primates, for example, baboons, monkeys, and chimpanzees may be used to generate BHLHB2 transgenic animals.

Any technique known in the art may be used to introduce the BHLHB2 transgene into animals to produce the founder lines of transgenic animals. Such techniques include, but are not limited to pronuclear microinjection (U.S. Pat. No. 4,873,191); retrovirus mediated gene transfer into germ lines (Van der Putten et al., Proc. Natl. Acad. Sci. USA 82:6148-6152, 1985); gene targeting in embryonic stem cells (Thompson et al., Cell 56:313-321, 1989); electroporation of embryos (Lo, Mol. Cell. Biol. 3:1803-1814, 1983); and sperm-mediated gene transfer (Lavitrano et al., Cell 57:717-723, 1989); etc. For a review of such techniques, see Gordon, Transgenic Animals, Intl. Rev. Cytol. 11.5:171-229, 1989, which is incorporated by reference herein in its entirety.

The present invention provides for transgenic animals that carry the BHLHB2 transgene in all their cells, as well as animals which carry the transgene in some, but not all their cells, i.e., mosaic animals. The transgene may be integrated as a single transgene or in concatamers, e.g., head-to-head tandems or head-to-tail tandems. The transgene may also be selectively introduced into and activated in a particular cell type by following, for example, the teaching of Lasko et al. (Lasko, M. et al., Proc. Natl. Acad. Sci. USA 89:6232-6236, 1992). Once transgenic animals have been generated, the expression of the recombinant BHLHB2 gene may be assayed utilizing standard techniques. Initial screening may be accomplished by Southern blot analysis or PCR techniques to analyze animal tissues to assay whether integration of the transgene has taken place. The level of mRNA expression of the transgene in the tissues of the transgenic animals may also be assessed using techniques which include but are not limited to Northern blot analysis of tissue samples obtained from the animal, in situ hybridization analysis, and RT-PCR. Samples of BHLHB2 gene-expressing tissue, may also be evaluated immunocytochemically using antibodies specific for the BHLHB2 transgene product.

Antibodies to BHLHB2 Proteins

Antibodies that specifically recognize one or more epitopes of BHLHB2 or peptide fragments of the BHLHB2 are also encompassed by the invention. Such antibodies include but are not limited to polyclonal antibodies, monoclonal antibodies (mAbs), humanized or chimeric antibodies, single chain antibodies, Fab fragments, F(ab′).2 fragments, fragments produced by a Fab expression library, anti-idiotypic (anti-Id) antibodies, and epitope-binding fragments of any of the above.

The antibodies of the invention may be used, for example, in the detection of the BHLHB2 in a biological sample and may, therefore, be utilized as part of a diagnostic or prognostic technique whereby patients may be tested for abnormal amounts of BHLHB2. Such antibodies may also be utilized in conjunction with, for example, compound screening schemes for the evaluation of the effect of test compounds on expression and/or activity of the BHLHB2 gene product. Additionally, such antibodies can be used in conjunction with the gene therapy techniques described, for example, evaluate the normal and/or engineered BHLHB2-expressing cells prior to their introduction into the patient. Such antibodies may additionally be used as a method for the inhibition of abnormal BHLHB2 activity. Thus, such antibodies may, therefore, be utilized as part of CAD treatment methods.

Methods of making and detecting labelled antibodies are well known (Campbell; Monoclonal Antibody Technology, in: Laboratory Techniques in Biochemistry and Molecular Biology, Volume 13. Eds: Burdon R et al. Elsevier, Amsterdam (1984)). The term antibody includes both monoclonal antibodies, which are a substantially homogeneous population, and polyclonal antibodies which are heterogeneous populations. The term also includes inter alia, humanised and chimeric antibodies. Monoclonal antibodies to specific antigens may be obtained by methods known to those skilled in the art, such as from hybridoma cells, phage display libraries or other methods. Monoclonal antibodies may be inter alia, human, rat or mouse derived. For the production of human monoclonal antibodies, hybridoma cells may be prepared by fusing spleen cells from an immunised animal, e.g. a mouse, with a tumour cell. Appropriately secreting hybridoma cells may thereafter be selected (Koehler & Milstein, Nature 256:495-497 (1975); Cole et al., “Monoclonal antibodies and Cancer Therapy”, Alan R Liss Inc, New York N.Y. pp 77-96 (1985)). Such antibodies may be of any immunoglobulin class including IgG, IgM, IgE, IgA, IgD and any subclass thereof.

Polyclonal antibodies can be generated by immunisation of an animal (such as a mouse, rat, goat, horse, sheep etc) with BHLHB2.

Rodent antibodies may be humanised using recombinant DNA technology according to techniques known in the art. Alternatively, chimeric antibodies, single chain antibodies, Fab fragments may also be developed against the polypeptides of the invention (Huse et al., Science 256:1275-1281 (1989)), using skills known in the art. Antibodies so produced have a number of uses which will be evident to the molecular biologist or immunologist skilled in the art. Such uses include, but are not limited to, monitoring enzyme expression, development of assays to measure enzyme activity and use as a therapeutic agent. Enzyme linked immunosorbant assays (ELISAs) are well known in the art and would be particularly suitable for detecting the BHLHB2 or polypeptide fragments thereof in a test sample.

Screening Assay

The invention also provides a method for identifying modulators, i.e., test compounds (e.g., peptides, peptidomimetics, small molecules or other drugs) which modulate the activity of the BHLHB2 gene or protein.

In one example, the invention provides methods for screening for test compounds for use in the treatment of CAD by screening for test compounds that modulate the activity of the BHLHB2 protein, or a portion thereof. The test compounds of the present invention can be obtained using any of the numerous approaches in combinatorial library methods known in the art, including: biological libraries. The method can be a cell-based method or a cell free method. The screening methods according to the invention may be operated using conventional procedures, for example by bringing the test compound or compounds to be screened and an appropriate substrate into contact with the BHLHB2 polypeptide, or a cell capable of producing it, or a cell membrane preparation thereof, and determining affinity for the BHLHB2 polypeptide in accordance with standard techniques.

Any compound identified in this way may prove useful in the treatment of CAD in humans and/or other animals. The invention thus extends to a compound selected through its ability to regulate the activity of the BHLHB2 protein in vivo as primarily determined in a screening assay utilising a BHLHB2 polypeptide or a homologue or fragment thereof, or a gene coding therefore for use in the treatment of a disease in which the over- or under-activity or unregulated activity of the protein is implicated.

According to a further aspect of the invention there is provided a screening assay or method for identifying potential CAD therapeutic compounds comprising contacting an assay system capable of detecting the effect of a test compound against expression level of BHLHB2, with a test compound and assessing the change in expression level of BHLHB2. Compounds that modulate the expression of DNA or RNA of the BHLHB2 polypeptide may be detected by a variety of assay systems. A suitable assay system may be a simple “yes/no” assay to determine whether there is a change in expression of a reporter gene, such as beta-galactosidase, luciferase, green fluorescent protein or others known to the person skilled in the art (reviewed by Naylor, Biochem. Pharmacol. 58:749-57, 1999). The assay system may be made quantitative by comparing the expression or function of a test sample with the levels of expression or function in a standard sample. Systems in which transcription factors are used to stimulate a positive output, such as transcription of a reporter gene, are generally referred to as “one-hybrid systems” (Wang, M. M. and Reed, R. R. Nature 364:121-126, 1993). Using a transcription factor to stimulate a negative output (growth inhibition) may thus be referred to as a “reverse one-hybrid system” (Vidal et al, 1996, supra). Therefore, in an embodiment of the present invention, a reporter gene is placed under the control of the BHLHB2 promoter.

In a further aspect of the invention a cell or cell line comprising a reporter gene under the control of the BHLHB2 promoter is provided.

According to another aspect of the present invention there is provided a method of screening for a compound potentially useful for treatment of CAD, which comprises assaying the compound for its ability to modulate the activity or amount of BHLHB2. Preferably the assay is selected from:
i) measurement of BHLHB2 activity using a cell line which expresses the BHLHB2 polypeptide or using purified BHLHB2 polypeptide; and
ii) measurement of BHLHB2 transcription or translation in a cell line expressing the BHLHB2 polypeptide.

Thus, in a further aspect of the invention, cell cultures expressing the BHLHB2 polypeptide can be used in a screen for therapeutic agents. Effects of test compounds may be assayed by changes in mRNA or protein of BHLHB2. As described above, cells (i.e. mammalian, bacterial, etc) can be engineered to express the BHLHB2 polypeptide.

Thus, according to a further aspect of the invention there is provided a method of testing potential therapeutic agents for the ability to suppress the CAD phenotype comprising contacting a test compound with a cell engineered to express the BHLHB2 polypeptide; and determining whether said test compound suppressed expression of the BHLHB2 polypeptide.

We also provide a method for identifying inhibitors of transcription of BHLHB2, which method comprises contacting a potential therapeutic agent with a cell or cell line as described above and determining inhibition of BHLHB2 transcription by the potential therapeutic agent by reference to a lack of or reduction in expression of the reporter gene.

Any convenient test compound or library of test compounds may be used in conjunction with the test assay. Particular test compounds include low molecular weight chemical compounds (preferably with a molecular weight less than 1500 daltons) suitable as pharmaceutical or veterinary agents for human or animal use, or compounds for non-administered use such as cleaning/sterilising agents or for agricultural use. Test compounds may also be biological in nature, such as antibodies.

According to a further aspect of the invention there is provided a compound identified by a screening method as defined herein.

According to another aspect of the present invention there is provided use of a compound able to modulate the activity or amount of BHLHB2 in the preparation of a medicament for the treatment of CAD. Modulation of the amount of BHLHB2 by a compound may be brought about for example through altered gene expression level or message stability. Modulation of the activity of BHLHB2 by a compound may also be brought about for example through compound binding to the BHLHB2 protein. In one embodiment, modulation of BHLHB2 comprises use of a compound able to reduce the activity or amount of BHLHB2. In another embodiment, modulation of BHLHB2 comprises use of a compound able to increase the activity or amount of BHLHB2.

Diagnostic Test

Methods for the diagnosis of CAD may, for example, utilize reagents such as the BHLHB2 nucleotide sequences and BHLHB2 antibodies. Specifically, such reagents may be used, for example, for: (1) the detection of the presence of BHLHB2 gene mutations, or the detection of either over- or under-expression of BHLHB2 mRNA relative to a control; (2) the detection of either an over- or an under-abundance of BHLHB2 gene product relative to a control; and (3) the detection of perturbations or abnormalities in the signal transduction pathway mediated by BHLHB2.

The methods described herein may be performed, for example, by utilizing pre-packaged diagnostic kits comprising at least one specific BHLHB2 nucleotide sequence or BHLHB2 antibody reagent described herein, which may be conveniently used, e.g., in clinical settings, to diagnose patients exhibiting body weight disorder abnormalities.

For the detection of BHLHB2 mutations, any nucleated cell can be used as a starting source for genomic nucleic acid. For the detection of BHLHB2 gene expression or BHLHB2 gene products, any cell type or tissue in which the BHLHB2 gene is expressed can be used.

Detection of BHLHB2 Polymorphisms

Knowledge of polymorphisms can be of assistance in identifying patients susceptible to particular diseases and those most suited to therapy with particular pharmaceutical agents (the latter is often termed “pharmacogenetics”). Pharmacogenetics can also be used in pharmaceutical research to assist the drug selection process. Polymorphisms are used in mapping the human genome and to elucidate the genetic component of diseases. The reader is directed to the following references for background details on pharmacogenetics and other uses of polymorphism detection: Linder et al. (1997), Clinical Chemistry, 43, 254; Marshall (1997), Nature Biotechnology, 15, 1249; International Patent Application WO 97/40462, Spectra Biomedical; and Schafer et al. (1998), Nature Biotechnology, 16, 33.

The present inventors have identified a number of polymorphisms within and around the BHLHB2 gene with demonstrable genetic association to coronary artery disease (CAD). The type and position of polymorphisms, which are identified in Table 3, are identified according to the position in Genbank ID:AB043885. Tables 4 and 5 provide sequence adjacent to the polymorphism site, which sequence can be used to unambiguously locate the position of the polymorphism in a nucleic acid sample, without reference to AB043885. SEQ ID Nos: 1-17 provide sequence of the polymorphism and sequence adjacent the polymorphism.

According to one aspect of the present invention there is provided a method for the diagnosis of a nucleotide polymorphism associated with CAD, which method comprises determining the sequence of the nucleotide at position 61 of any one of SEQ ID NOs: 1 to 5, 7, 8 and 10 to 17, or determining the number of AT repeats starting at position 61 of SEQ ID NO: 6, or the number of AC repeats starting at position 63 of SEQ ID NO: 6, or determining the combined number of AT and AC repeats starting at position 61 of SEQ ID NO: 6, or the number of GT repeats starting at position 61 of SEQ ID NO: 9, and determining the status of the human by reference to polymorphism(s) detected.

The inventors have found that there are 4-7 AT repeats starting at position 61 of SEQ ID NO:6; 8-15 AC repeats staring at position 63 of SEQ ID NO: 6. Various combinations of AT and AC repeats have been found (see later). The inventors have also found that there are 12-22 GT repeats starting at position 61 of SEQ ID NO: 9. In view of the functionality restrictions on including repeat units with Patentin software, both SEQ ID NO: 6 and SEQ ID NO: 9 have been written as if there are no repeats present.

The term human includes both a human having or suspected of having inflammatory bowel disease and an asymptomatic human who may be tested for predisposition or susceptibility to CAD. At each position the human may be homozygous for an allele or the human may be a heterozygote.

In one embodiment of the invention preferably the method for diagnosis described herein is one in which the single nucleotide polymorphism at position 61 (according to SEQ ID NO: 1) is the presence of G and/or A.

In another embodiment of the invention preferably the method of diagnosis described herein is one in which the single nucleotide polymorphism at position 61 (according to SEQ ID NO: 2) is the presence of A and/or C.

In another embodiment of the invention preferably the method of diagnosis described herein is one in which the single nucleotide polymorphism at position 61 (according to SEQ ID NO: 3) is the presence of C and/or T.

In another embodiment of the invention preferably the method of diagnosis described herein is one in which the single nucleotide polymorphism at position 61 (according to SEQ ID NO: 4) is the presence of C and/or T.

In another embodiment of the invention preferably the method of diagnosis described herein is one in which the single nucleotide polymorphism at position 61 (according to SEQ ID NO: 5) is the presence of C and/or T.

In another embodiment of the invention preferably the method of diagnosis described herein is one in which the number of AT repeats starting at position 61 (according to SEQ ID NO: 6) is determined. In various embodiments, this number is from 4 and 7.

In another embodiment of the invention preferably the method of diagnosis described herein is one in which the number of CA repeats starting at position 63 (relative to the position in SEQ ID NO: 6) is determined. In various embodiments, this number is from 8 and 15.

In another embodiment of the invention preferably the method of diagnosis described herein is one in which the combined number of AT and CA repeats starting at position 61 (according to SEQ ID NO: 6) is determined. In various embodiments, this number is from 13 and 21, inclusive.

In another embodiment of the invention preferably the method of diagnosis described herein is one in which the single nucleotide polymorphism at position 61 (according to SEQ ID NO: 7) is the presence of T and/or C.

In another embodiment of the invention preferably the method of diagnosis described herein is one in which the single nucleotide polymorphism at position 61 (according to SEQ ID NO: 8) is the presence of A and/or G.

In another embodiment of the invention preferably the method of diagnosis described herein is one in which the number of GT repeats starting at position 61 (according to SEQ ID NO: 9) is determined. In various embodiments the number of repeats is between 12 and 22, inclusive.

In another embodiment of the invention preferably the method of diagnosis described herein is one in which the single nucleotide polymorphism at position 61 (according to SEQ ID NO: 10) is the presence of G and/or C.

In another embodiment of the invention preferably the method of diagnosis described herein is one in which the single nucleotide polymorphism at position 61 (according to SEQ ID NO: 10) is the presence of G and/or T.

In another embodiment of the invention preferably the method of diagnosis described herein is one in which the single nucleotide polymorphism at position 61 (according to SEQ ID NO: 12) is the presence of G and/or A.

In another embodiment of the invention preferably the method of diagnosis described herein is one in which the single nucleotide polymorphism at position 61 (according to SEQ ID NO: 13) is the presence of A and/or G.

In another embodiment of the invention preferably the method of diagnosis described herein is one in which the single nucleotide polymorphism at position 61 (according to SEQ ID NO: 14) is the presence of A and/or T.

In another embodiment of the invention preferably the method of diagnosis described herein is one in which the single nucleotide polymorphism at position 61 (according to SEQ ID NO: 15) is the presence of A and/or C.

In another embodiment of the invention preferably the method of diagnosis described herein is one in which the single nucleotide polymorphism at position 61 (according to SEQ ID NO: 16) is the presence of G and/or A.

In another embodiment of the invention preferably the method of diagnosis described herein is one in which the single nucleotide polymorphism at position 61 (according to SEQ ID NO: 17) is the presence of A and/or T.

The method for diagnosis is preferably one in which the sequence is determined by a method selected from amplification refractory mutation system and restriction fragment length polymorphism.

In another aspect of the invention we provide a method for the diagnosis or prognosis or inflammatory bowel disease, which method comprises:
i) obtaining sample nucleic acid from an individual,
ii) detecting the presence or absence of a variant nucleotide at position 61 relative to one or more of the sequences disclosed in SEQ ID NO: 1 to 5, 7, 8 and 10 to 17, or detecting the number of AT repeats starting at position 61 of SEQ ID NO: 6, or the number of AC repeats starting at position 63 of SEQ ID NO: 6, or the combined number of AT and AC repeats starting at position 61 of SEQ ID NO: 6, or the number of GT repeats starting at position 61 of SEQ ID NO: 9; and,
iii) determining the status of the individual by reference to the particular variant polymorphism.

The test sample of nucleic acid is conveniently a sample of blood, bronchoalveolar lavage fluid, sputum, or other body fluid or tissue obtained from an individual. It will be appreciated that the test sample may equally be a nucleic acid sequence corresponding to the sequence in the test sample, that is to say that all or a part of the region in the sample nucleic acid may firstly be amplified using any convenient technique e.g. PCR, before use in the analysis of BHLHB2 variation.

It will be apparent to the person skilled in the art that there are a large number of analytical procedures, which may be used to detect the presence or absence of the various SNPs of the invention. In general, the detection of allelic variation requires a mutation discrimination technique, optionally an amplification reaction and a signal generation system. Table 1 lists a number of mutation detection techniques, some based on the PCR. These may be used in combination with a number of signal generation systems, a selection of which is listed in List 1. Further amplification techniques are listed in List 2. Many current methods for the detection of allelic variation are reviewed by Nollau et al., Clin. Chem. 43, 1114-1120, 1997; and in standard textbooks, for example “Laboratory Protocols for Mutation Detection”, Ed. by U. Landegren, Oxford University Press, 1996 and “PCR”, 2^{nd} Edition by Newton & Graham, BIOS Scientific Publishers Limited, 1997.

Abbreviations:

| | |
|---|---|
| ALEX ™ | Amplification refractory mutation system linear extension |
| APEX | Arrayed primer extension |
| ARMS ™ | Amplification refractory mutation system |
| b-DNA | Branched DNA |
| CMC | Chemical mismatch cleavage |
| Bp | base pair |
| COPS | Competitive oligonucleotide priming system |
| DGGE | Denaturing gradient gel electrophoresis |
| FRET | Fluorescence resonance energy transfer |
| LCR | Ligase chain reaction |
| MASDA | Multiple allele specific diagnostic assay |
| NASBA | Nucleic acid sequence based amplification |
| OLA | Oligonucleotide ligation assay |
| PCR | Polymerase chain reaction |
| PTT | Protein truncation test |
| RFLP | Restriction fragment length polymorphism |
| SDA | Strand displacement amplification |
| SNP | Single nucleotide polymorphism |
| SSCP | Single-strand conformation polymorphism analysis |
| SSR | Self sustained replication |
| TGGE | Temperature gradient gel electrophoresis |

TABLE 1
Mutation Detection Techniques

General: DNA sequencing, Sequencing by hybridisation
Scanning: PTT*, SSCP, DGGE, TGGE, Cleavase, Heteroduplex analysis,
CMC, Enzymatic mismatch cleavage
Hybridisation Based
Solid phase hybridisation: Dot blots, MASDA, Reverse dot blots,
Oligonucleotide
arrays (DNA Chips)
Solution phase hybridisation: Taqman ™ - US-5210015 & US-5487972
(Hoffmann-La
Roche), Molecular Beacons - Tyagi et al (1996), Nature Biotechnology,
14, 303; WO
95/13399 (Public Health Inst., New York)
Extension Based: ARMS ™, ALEX ™ - European Patent No.
EP 332435 B1 (Zeneca Limited), COPS - Gibbs et al (1989),
Nucleic Acids Research, 17, 2347.
Incorporation Based: Mini-sequencing, APEX
Restriction Enzyme Based: RFLP, Restriction site generating PCR
Ligation Based: OLA
Other: Invader assay
*Note: not useful for detection of promoter polymorphisms.

List 1—Signal Generation or Detection Systems
Fluorescence: FRET, Fluorescence quenching, Fluorescence polarisation—United Kingdom Patent No. 2228998 (Zeneca Limited)
Other: Chemiluminescence, Electrochemiluminescence, Raman, Radioactivity, Colorimetric, Hybridisation protection assay, Mass spectrometry
List 2—Further Amplification Methods
SSR, NASBA, LCR, SDA, b-DNA

Preferred mutation detection techniques include ARMS™, ALEX™, COPS, Taqman, Molecular Beacons, RFLP, and restriction site based PCR and FRET techniques.

Particularly preferred methods include Taqman, ARMS™ and RFLP based methods. Taqman is an especially preferred method.

Assays, for example reporter-based assays, may be devised to detect whether one or more of the above polymorphisms affect transcription levels.

In a further aspect, the diagnostic methods of the invention, are used to assess the predisposition and/or susceptibility of an individual to CAD, and the present invention may be used to recognise individuals who are particularly at risk from developing CAD conditions.

In a further aspect, the diagnostic methods of the invention are used in the development of new drug therapies, which selectively target one or more allelic variants identified herein. Identification of a link between a particular allelic variant and predisposition to disease development or response to drug therapy may have a significant impact on the design of new drugs. Drugs may be designed to regulate the biological activity of variants implicated in the disease process whilst minimising effects on other variants.

In a further diagnostic aspect of the invention the presence or absence of variant nucleotides is detected by reference to the loss or gain of, optionally engineered, sites recognised by restriction enzymes. The person of ordinary skill will be able to design and implement diagnostic procedures based on the detection of restriction fragment length polymorphism due to the loss or gain of one or more of the sites.

The invention further provides nucleotide primers which detect the polymorphisms of the invention.

The invention further provides nucleotide probes which can detect the polymorphisms of the invention.

According to another aspect of the present invention a person of ordinary skill will be able to design allele specific primers or probes capable of detecting a polymorphism at position 61 at of any one of SEQ ID NOs: 1 to 5, 7, 8 and 10 to 17, or a variant combined number of AT and AC repeats starting at position 61 of SEQ ID NO: 6, or a variant number of GT repeats starting at position 61 of SEQ ID NO: 9.

An allele specific primer is used, generally together with a constant primer, in an amplification reaction such as a PCR reaction, which provides the discrimination between alleles through selective amplification of one allele at a particular sequence position e.g. as used for ARMS™ assays. The allele specific primer is preferably 17-50 nucleotides, more preferably about 17-35 nucleotides, more preferably about 17-30 nucleotides. An allele specific primer preferably corresponds exactly with the allele to be detected but derivatives thereof are also contemplated wherein about 6-8 of the nucleotides at the 3′ terminus correspond with the allele to be detected and wherein up to 10, such as up to 8, 6, 4, 2, or 1 of the remaining nucleotides may be varied without significantly affecting the properties of the primer.

Primers may be manufactured using any convenient method of synthesis. Examples of such methods may be found in standard textbooks, for example “Protocols for Oligonucleotides and Analogues; Synthesis and Properties,” Methods in Molecular Biology Series; Volume 20; Ed. Sudhir Agrawal, Humana ISBN: 0-89603-247-7; 1993; 1^{st} Edition. If required the primer(s) may be labelled to facilitate detection.

According to another aspect of the present invention there is provided an allele-specific oligonucleotide probe capable of detecting a polymorphism at position 61 of any one of SEQ ID NOs: 1 to 5, 7, 8 and 10 to 17, or a variant number of CA repeats starting at position 61 of SEQ ID NO: 6, or a variant number of GT repeats starting at position 61 of SEQ ID NO: 9.

The allele-specific oligonucleotide probe is preferably 17-50 nucleotides, more preferably about 17-35 nucleotides, more preferably about 17-30 nucleotides. Representative examples of oligonucleotide probes are those identified in SEQ ID NO: 1 to 17, but wherein the nucleotide at position 31 is one or other of the polymorphic alleles.

The design of such probes will be apparent to the molecular biologist of ordinary skill. Such probes are of any convenient length such as up to 50 bases, up to 40 bases, more conveniently up to 30 bases in length, such as for example 8-25 or 8-15 bases in length. In general such probes will comprise base sequences entirely complementary to the corresponding wild type or variant locus in the gene. However, if required one or more mismatches may be introduced, provided that the discriminatory power of the oligonucleotide probe is not unduly affected. The probes of the invention may carry one or more labels to facilitate detection.

According to another aspect of the present invention there is provided a diagnostic kit comprising a diagnostic primer of the invention and/or an allele-specific oligonucleotide primer of the invention.

The diagnostic kits may comprise appropriate packaging and instructions for use in the methods of the invention. Such kits may further comprise appropriate buffer(s) and polymerase(s) such as thermostable polymerases, for example taq polymerase.

In another aspect of the invention, the single nucleotide polymorphisms of this invention may be used as genetic markers for this region in linkage studies.

Detection of the CAD Gene and Transcripts

BHLHB2 gene transcripts and mutations within the BHLHB2 gene can be detected by utilizing a number of techniques. Nucleic acid from any nucleated cell can be used as the starting point for such assay techniques, and may be isolated according to standard nucleic acid preparation procedures which are well known to those of skill in the art.

DNA may be used in hybridization or amplification assays of biological samples to detect abnormalities involving BHLHB2 gene structure or gene regulatory elements, including point mutations, insertions, deletions and chromosomal rearrangements. Such assays may include, but are not limited to, Southern analyses, single stranded conformational polymorphism analyses (SSCP), and PCR analyses.

Such diagnostic methods for the detection of BHLHB2 gene-specific mutations can involve for example, contacting and incubating nucleic acids including recombinant DNA molecules, cloned genes or degenerate variants thereof, obtained from a sample, for example, derived from a patient sample or other appropriate cellular source, with one or more labeled nucleic acid reagents including recombinant DNA molecules, under conditions favorable for the specific annealing of these reagents to their complementary sequences within the BHLHB2 gene. Preferably, the lengths of these nucleic acid reagents are at least 15 to 30 nucleotides. After incubation, all non-annealed nucleic acids are removed from the nucleic acid: BHLHB2 molecule hybrid. The presence of nucleic acids which have hybridized, if any such molecules exist, is then detected. Using such a detection scheme, the nucleic acid from the cell type or tissue of interest can be immobilized, for example, to a solid support such as a membrane, or a plastic surface such as that on a microtiter plate or polystyrene beads. Detection of the remaining, annealed, labeled BHLHB2 nucleic acid reagents is accomplished using standard techniques well-known to those in the art. The BHLHB2 gene sequences to which the nucleic acid reagents have annealed can be compared to the annealing pattern expected from a normal BHLHB2 gene sequence in order to determine whether a BHLHB2 gene mutation is present.

Detection of the BHLHB2 Gene Products

Antibodies directed against wild type or mutant BHLHB2 gene products or conserved variants or peptide fragments thereof, can also be used as CAD diagnostics. Such diagnostic methods, can be used to detect abnormalities in the level of BHLHB2 gene expression, or abnormalities in the structure and/or temporal, tissue, cellular, or subcellular location of the BHLHB2, and may be performed in vivo or in vitro, such as, for example, on biopsy tissue.

For example, antibodies directed to epitopes of the BHLHB2 can be used in vivo to detect the pattern and level of expression of the BHLHB2 in the body. Such antibodies can be labeled, and injected into a subject in order to visualize binding to the BHLHB2 expressed in the body using methods such as X-rays, CAT-scans, or MRI.

Additionally, any BHLHB2 fusion protein or BHLHB2 conjugated protein whose presence can be detected, can be administered. For example, BHLHB2 fusion or conjugated proteins labeled with a radio-opaque or other appropriate compound can be administered and visualized in vivo, as discussed, above for labeled antibodies.

Alternatively, immunoassays or fusion protein detection assays, as described above, can be utilized on biopsy and autopsy samples in vitro to permit assessment of the expression pattern of the BHLHB2.

The tissue or cell type to be analyzed will generally include those which are known, or suspected, to express the BHLHB2 gene. The protein isolation methods employed herein can, for example, be such as those described in Harlow and Lane (Harlow, E. and Lane, D, Antibodies: A Laboratory Manual, Cold Spring Harbor Laboratory Press, Cold Spring Harbor, N.Y., 1989), which is incorporated herein by reference in its entirety. The isolated cells can be derived from cell culture or from a patient. The analysis of cells taken from culture may be a necessary step in the assessment of cells that could be used as part of a cell-based gene therapy technique or, alternatively, to test the effect of compounds on the expression of the BHLHB2 gene.

Gene Therapy

The invention encompasses gene therapy methods and compositions for treating and preventing CAD.

In one example, the loss of normal BHLHB2 gene product function can result in the development of CAD. Increasing BHLHB2 gene product activity, or activation of the BHLHB2 pathway (e.g., downstream activation) would therefore facilitate progress in individuals exhibiting a deficient level of BHLHB2 gene expression and/or BHLHB2 activity.

Alternatively, CAD may be ameliorated by decreasing the level of BHLHB2 gene expression, and/or BHLHB2 gene activity, and/or downregulating activity of the BHLHB2 pathway (e.g., by targeting downstream signaling events). Different approaches are discussed below.

Inhibition of BHLHB2 Expression or BHLHB2 Activity

Any method that neutralizes or inhibits expression of the BHLHB2 gene (either transcription or translation) can be used to prevent or treat CAD.

For example, the administration of soluble peptides, proteins, fusion proteins, or antibodies (including anti-idiotypic antibodies) that bind to and “neutralize” circulating BHLHB2 can be used. Such BHLHB2 neutralizing peptides, proteins, fusion proteins, anti-idiotypic antibodies or Fabs are administered to a subject in amounts sufficient to treat or prevent CAD.

In an alternate embodiment, therapy can be designed to reduce the level of endogenous BHLHB2 gene expression, for example, using antisense or ribozyme approaches to inhibit or prevent translation of BHLHB2 mRNA-transcripts; triple helix approaches to inhibit transcription of the BHLHB2 gene; or targeted homologous recombination to inactivate or “knock out” the BHLHB2 gene or its endogenous promoter. Antisense, ribozyme or DNA constructs can be administered directly to the site containing the target cells; or can be directed to the target cells.

Antisense approaches involve the design of oligonucleotides (either DNA or RNA) that are complementary to BHLHB2 mRNA. The antisense oligonucleotides will bind to the complementary BHLHB2 mRNA transcripts and prevent translation. Absolute complementarity, although preferred, is not required. A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, for example, the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. Thus, oligonucleotides complementary to either the 5′- or 3′-non-translated, non-coding regions of the BHLHB2 can be used in an antisense approach to inhibit translation of endogenous BHLHB2 mRNA. Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of BHLHB2 mRNA, antisense nucleic acids should be at least six nucleotides in length, and are preferably oligonucleotides ranging from 6 to about 50 nucleotides in length. In specific aspects, the oligonucleotide is at least 10 nucleotides, at least 17 nucleotides, at least 25 nucleotides, or at least 50 nucleotides in length.

Regardless of the choice of target sequence, it is preferred that in vitro studies are first performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

While antisense nucleotides complementary to the BHLHB2 coding region sequence could be used, those complementary to the transcribed untranslated region are most preferred. The antisense molecules should be delivered to cells which express the BHLHB2 in vivo. A number of methods have been developed for delivering antisense DNA or RNA to cells; for example, antisense molecules can be injected directly into the tissue site, or modified antisense molecules, designed to target the desired cells (e.g., antisense linked to peptides or antibodies that specifically bind receptors or antigens expressed on the target cell surface) can be administered systemically.

Ribozyme molecules designed to catalytically cleave BHLHB2 mRNA transcripts can also be used to prevent translation of BHLHB2 mRNA and expression of BHLHB2. (See, e.g., WO90/11364; Sarver et al., Science 247:1222-1225, 1990). While ribozymes that cleave mRNA at site specific recognition sequences can be used to destroy BHLHB2 mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, Nature 334:585-591, 1988. There are hundreds of potential hammerhead ribozyme cleavage sites within the nucleotide sequence of human BHLHB2 cDNA. Preferably the ribozyme is engineered so that the cleavage recognition site is located near the 5′ end of the BHLHB2 mRNA; i.e., to increase efficiency and minimize the intracellular accumulation of non-functional mRNA transcripts.

The ribozymes of the present invention also include RNA endoribonucleases (hereinafter “Cech-type ribozymes”) such as the one which occurs naturally in Tetrahymena Thermophila (known as the IVS, or L-19 IVS RNA) and which has been extensively described by Thomas Cech and collaborators (Zaug, et al., Science 224:574-578, 1984; Zaug and Cech, Science 231:470-475, 1986; Zaug, et al., Nature 324:429-433, 1986; patent application No. WO 88/04300; Been and Cech, Cell 47:207-216, 1986). The Cech-type ribozymes have an eight basepair active site that hybridizes to a target RNA sequence, whereafter cleavage of the target RNA takes place. The invention encompasses those Cech-type ribozymes that target eight basepair active site sequences that are present in BHLHB2.

As in the antisense approach, the ribozymes can be composed of modified oligonucleotides (e.g. for improved stability, targeting, etc.) and should be delivered to cells which express the BHLHB2 in vivo. A preferred method of delivery involves using a DNA construct “encoding” the ribozyme under the control of a strong constitutive pol III or pol II promoter, so that transfected cells will produce sufficient quantities of the ribozyme to destroy endogenous BHLHB2 messages and inhibit translation. Because ribozymes, unlike antisense molecules, are catalytic, a lower intracellular concentration is required for efficiency.

In an alternative embodiment for neutralizing circulating BHLHB2, cells that are genetically engineered to express such soluble or secreted forms of BHLHB2 can be administered to a patient, whereupon they will serve as “bioreactors” in vivo to provide a continuous supply of the neutralizing protein.

Endogenous BHLHB2 gene expression can also be reduced by inactivating or “knocking out” the BHLHB2 gene or its promoter using targeted homologous recombination. (E.g., see Smithies et al., Nature 317:230-234, 1985; Thomas & Capecchi, Cell 51:503-512, 1987; Thompson et al., Cell 5:313-321, 1989; each of which is incorporated by reference herein in its entirety).

In yet another embodiment of the invention, the activity of BHLHB2 can be reduced using a “dominant negative” approach to effectuate BHLHB2. To this end, constructs that encode defective BHLHB2s can be used in gene therapy approaches to diminish the activity of the BHLHB2 in appropriate target cells.

Restoration or Increase in BHLHB2 Expression or Activity

With respect to an increase in the level of BHLHB2 gene expression and/or BHLHB2 gene product activity, BHLHB2 nucleic acid sequences can be utilized for the treatment of CAD. Where the cause of CAD is a defective BHLHB2 gene, treatment can be administered, for example, in the form of gene replacement therapy. Specifically, one or more copies of a normal BHLHB2 gene or a portion of the BHLHB2 gene that directs the production of a BHLHB2 gene product exhibiting normal function, can be inserted into the appropriate cells within a patient or animal subject, using vectors which include, but are not limited to, adenovirus, adeno-associated virus, retrovirus, and herpes virus vectors, in addition to other particles that introduce DNA into cells, such as liposomes.

Additional methods that can be utilized to increase the overall level of BHLHB2 gene expression and/or BHLHB2 activity include the introduction of appropriate BHLHB2-expressing cells, preferably autologous cells, into a patient at positions and in numbers which are sufficient to ameliorate the symptoms of CAD. Such cells can be either recombinant or non-recombinant. Among the cells that can be administered to increase the overall level of BHLHB2 gene expression in a patient are normal cells which express the BHLHB2 gene. The cells can be administered to the site of interest. Such cell-based gene therapy techniques are well known to those skilled in the art, see, for example, Anderson, et al., U.S. Pat. No. 5,399,349; Mulligan & Wilson, U.S. Pat. No. 5,460,959.

Finally, compounds, identified in the assays described above, that stimulate or enhance the signal transduced by activated BHLHB2, for example, by activating downstream signalling proteins in the BHLHB2 cascade, and thereby by-passing the defective BHLHB2. The formulation and mode of administration will depend upon the physico-chemical properties of the compound.

Pharmaceutical Composition Containing the Identified Candidate Compound

The compounds having the desired activity may be administered in a physiologically acceptable carrier to a CAD patient. Such compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal track, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Suppository formulations may be prepared by mixing the active ingredient with a suitable non-irritating excipient which is solid at ordinary temperatures but liquid at the rectal temperature and will therefore melt in the rectum to release the drug. Suitable excipients include, for example, cocoa butter and polyethylene glycols.

Topical formulations, such as creams, ointments, gels and aqueous or oily solutions or suspensions, may generally be obtained by formulating an active ingredient with a conventional, topically acceptable, vehicle or diluent using conventional procedure well known in the art.

Compositions for administration by insufflation may be in the form of a finely divided powder containing particles of average diameter of, for example, 30μ or much less, the powder itself comprising either active ingredient alone or diluted with one or more physiologically acceptable carriers such as lactose. The powder for insufflation is then conveniently retained in a capsule containing, for example, 1 to 50 mg of active ingredient for use with a turbo-inhaler device, such as is used for insufflation of the known agent sodium cromoglycate.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on Formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

The size of the dose for therapeutic or prophylactic purposes of a compound will naturally vary according to the nature and severity of the conditions, the age and sex of the animal or patient and the route of administration, according to well known principles of medicine.

In using a compound for therapeutic or prophylactic purposes it will generally be administered so that a daily dose in the range, for example, 0.5 mg to 75 mg per kg body weight is received, given if required in divided doses. In general lower doses will be administered when a parenteral route is employed. Thus, for example, for intravenous administration, a dose in the range, for example, 0.5 mg to 30 mg per kg body weight will generally be used. Similarly, for administration by inhalation, a dose in the range, for example, 0.5 mg to 25 mg per kg body weight will be used. Oral administration is however preferred.

It will be obvious to those skilled in the art to which the invention pertains, that various changes and modifications may be made without departing from the scope of the invention defined by the claims.

### EXAMPLE 1

530 affected sib-pairs (ASPs) from 407 families with both sibs having CAD before 65 years were collected from hospitals in Sweden, Italy and Germay. All ASPs were recruited either from hospital discharge databases, existing research-based patient databases or from coronary care units.

Probands were individuals with documented myocardial infarction (MI) or other symptomatic acute coronary syndrome (ACS; defined as hospitalisation for one of the following indications: a) unstable angina; b) thrombolysis for suspected MI (localised ST-elevation in 2 or more ECG leads); c) emergency revascularisation for control of typical ischaemic chest pain at rest) first diagnosed before 65 years and confirmed by the physician. Affected sibs were individuals with confirmed MI, other symptomatic ACS as for the proband but in addition, chronic stable angina first diagnosed before 65 years and confirmed by physician, was included.

Preparation of Genomic DNA from Blood Cells

Blood samples were stored at −80° C. Before extraction the samples were thawed at 37° C. in a water bath. To extract the genomic DNA, using the PUREGENE DNA Isolation kit from Gentra Systems, the blood cells were added to 30 ml of RBC lysis solution in a 50 ml centrifuge tube. The mixture was inverted twice while incubating for 10 minutes (min) at room temperature (RT). To separate the white cells from other lysed material, the mixture was centrifuged in a RT-7™ from Sorvall Ltd. at 2,000×g for 10 min. Most of the supernatant was removed leaving approximately 100 ml of residual solution to resuspend the cells in by vigorously votexing. Following resuspension 10 ml of cell lysis solution were added and mixed by pipetting. To digest any RNA, 50 ml of RNase A solution were added to the lysate, which was then incubated at 37° C. for 15 min. After RNA digestion, 3.3 ml of protein precipitation solution was added and the mixture was vortexed for 20 seconds (s). To separate the proteins from the DNA, the mixture was again centrifuged at 2,000×g for 10 min. The supernatant retaining the DNA was then poured off into another 50 ml centrifuge tube containing 10 ml of 100% isopropanol and mixed by inverting 50×. To separate the DNA from the aqueous solution, it was removed with an inoculating loop and washed in 10 ml 70% ethanol in a 50 ml centrifuge tube. to remove any residual salt. The DNA was removed from the 70% ethanol using an inoculating loop and air dried briefly. To hydrate, the DNA was added to 1 ml of DNA hydration solution in a 14 ml centrifuge tube and this was rocked at RT overnight allowing the DNA to equilibrate. The resulting DNA samples were transferred to 1.5 ml tubes and stored at −80° C. The concentration of extracted genomic DNA was quantified using the Fluorocount™ from Packard Instruments Co. The DNA was diluted to 4 ng/ml into a working plate.

Dispensing DNA from a working plate into several 96-well plates for subsequent polymerase chain reaction (PCR) amplification reactions was performed using the Hydra™ 96 from Robbins Scientific, Inc. The Hydra™ was programmed to dispense 5 ml of DNA from each well of a ‘working plate’ into the corresponding wells of the required number of PCR plates. Before dispensing, the Hydra™ was washed with water. Following dispensing the Hydra™ was sterilised by washing with water, with 2% chloros industrial, and again with water. The resulting PCR plates, containing 20 ng of DNA in each well, were dried at 80° C. for 1 hr. The plates were stored at −20° C.

Microsatellite Genotyping for Genome Scan

A total of 384 microsatellite markers were genotyped across the CAD ASP families. The majority of these were from the linkage mapping set 2.0 (Applied Biosystems). Additional markers were included to replace those excluded by error checking procedures. The amplification of microsatellite markers by PCR and subsequent pooling of the amplified products into panels were carried out using a RapidGene™ automated system from Oxagen Ltd. The PCR plates contained 20 ng of dried genomic DNA in each well. The 1.5 ml tubes contained 5 mM of appropriate ‘forward’ oligonucleotide, 5 mM of the corresponding ‘reverse’ oligonucleotide, 1×PCR buffer II, 2.5 mM MgCl2, 1 mM dNTP, and 0.06 u/ml AmpliTaq Gold™ DNA polymerase in final volume of 1.2 ml. During the run, 10 ml of the appropriate PCR reaction mixture were dispensed into the wells of the plates. Following amplification the reactions were pooled into panels and the pool plates were sealed and stored at 4° C. Pooled DNA fragments generated from the amplification of microsatellite markers were separated by capillary electrophoresis using an ABI 3700 automated sequencer (PE Applied Biosystems, Inc.). Products were sized using the programme ABI GENESCAN (version 3.0) (PE Biosystems) and genotypes assigned semi-automatically using the GENOTYPER software (version 2.0).

Linkage Analysis Chromosome 3

In a genome wide screen with 384 markers (average marker distance 9.2 cm) 530 affected sib-pairs were analysed from 407 families. In addition marker genotypes for at least one parent or unaffected sibs were available in 110 families.

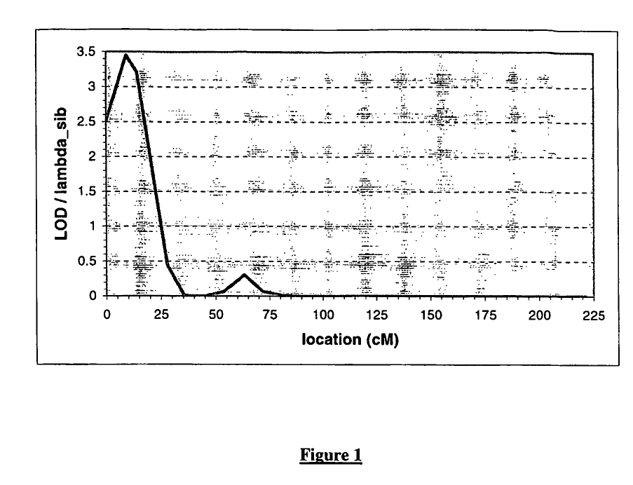

The genotype data for chromosome 3 include 23 markers (see FIG. 1) with an average distance of 9.8 cM. Linkage analysis was performed in Genehunter-Plus version 1.3 (Kruglyak et al, Am. J. Hum. Genet. 58, 1347-1363 (1996) and Kruglyak and Lander. Journal of Computational Biology 5:1-7 (1998)) using the ‘all’ scoring function, followed by calculation of LOD-scores in KAC as described by Kong and Cox Am. J. Hum. Genet. 61, 1179-1188 (1997). A maximum LOD-score of 3.45 was obtained at 9 cM, with the position given as the distance from the most telomeric marker D3S1297 (FIG. 1). In table 2, LOD-scores for the three markers overlapping the peak are displayed.

| Position | LOD | Marker |
|---|---|---|
| 0.0 | 2.51 | D3S1297 |
| 9.0 | 3.45 | — |
| 14.1 | 3.21 | D3S1304 |
| 27.8 | 0.45 | D3S1263 |

To assess the statistical significance of the obtained LOD-score we simulated marker genotype data under a model with no genetic effect, preserving the family structures and missing data patterns of the families in the study, using the observed marker allele frequencies. In 3600 replicates no LOD-scores as large as the one observed for the real data was observed for chromosome 3, indicating a p-value less than 0.001. Consideration of the LOD-scores obtained on all autosomal chromosomes in each replicate resulted in an empirical genome-wide p-value of 0.01.

Genetic marker distances were obtained from the Marshfield map and translated to recombination fractions using the Kosambi mapping function. The marker allele frequencies were estimated from all genotyped individuals in the study.

Prior to the linkage analysis the genotype data went through quality control. This includes identification of half-sibs or unrelated individuals among the putative sib pairs using RELPAIR (Boehnke et al., Am. J. Hum. Genet. 61:423-429. 1997), detection of Mendelian inconsistencies with PEDCHECK (O'Connell et al., Am. J. Hum. Genet. 63:259-266 1998) and retyping of inconsistent marker-family combinations. Detected half-sib pairs were excluded from the linkage analysis described here.

Associated Gene Identification

The highest linkage was found between makers D3S1297 and D3S1304, within this region lies the BHLHB2 gene. Further SNP mapping identified this gene as being associated with the CAD.

### EXAMPLE 2

Identification of Polymorphisms in BHLHB2, a Coronary Artery Disease (CAD) Susceptibility Gene on Chromosome 3p

According to the Ensembl genome assembly (build 31), the BHLHB2 gene consists of 5 exons covering a genomic distance of 5.7 kb. BLAST analysis using BHLHB2 cDNA suggests that there might be one or two additional 5′ untranslated exons. The gene was screened for polymorphisms, and a selection of the idenitfied polymorphisms were subsequently tested for association to CAD in a TDT material.

1. DNA Sequencing of BHLHB2 Exons 1-5

Individual BHLHB2 exons were PCR amplified from genomic DNA of 24 unrelated individuals, using oligonucleotide primers designed in flanking intronic sequences.

The PCR amplified products were then sequenced and SNPs identified by Dye-primer sequencing as described in the ABI protocol P/N 402114 using ABI 3700 automated sequencers. Genotyping of informative SNPs was performed by real-time PCR using the TAQMAN™ technology from PE Biosystems.

The SNPs (or other polymorphisms) and their frequencies are shown in Table 3. Sequence flanking the polymorphisms is shown in Table 4, Table 5 shows sequence adjacent the polymorphisms.

2. Mining Public SNP Databses

Additional public domain SNPs in the BHLHB2 gene were identified (dbSNP; http://www.ncbi.nlm.nih.gov/SNP/).

SNP Genotyping

Five SNPs discovered in Step 1 (rs1110261, rs2271566, rs2671757, rs908078, and DEC1az 1494), were genotyped in at least 92 UK blood donor control DNAs obtained for validation of SNPs in the TDT collection. The results are shown in Table 3.

Microsatelite Genotyping

One marker discovered in Step 1, DEC1azGTrep, was genotyped in a number of individuals from the TDT collection. The results are shown in Table 3.

TABLE 3

| | | | | | |
|---|---|---|---|---|---|
| | | Position in gene | | | “Single” p- |
| Internal | | (Genbank ID: | | Allele frequency | value in fist |
| identifier | Identifier | AB043885) | *Allele change | (# of individuals) | TDT analysis |

| | | | | | |
|---|---|---|---|---|---|
| 1 | rs1110261 | 2176 bp | G/A | no info | 0.7362 |
| 2 | rs1104976 | 2388 bp | A/C | unconfirmed | |
| 3 | rs2271566 | 3643 bp | C/T | C = 84% / T = 16% | 0.9943 |
| | | | (3300) | | |
| 4 | az0001116 | 3944 bp | C/T/- | not polymorphic | |
| 5 | rs2671757 | 5112 bp | C/T | C = 81% / T = 19% | 0.0087 |
| | | | (3304) | | |
| 6 | DEC1azCArep | 5112-5133 bp | (AT)n(CA)m | * | |
| 7 | rs908078 | 7002 bp | T/C | T = 84% / C = 16% | 0.7346 |
| | | | | (3268) | |
| 8 | rs3205438 | 7082 bp | A/G | not polymorphic | |
| 9 | DEC1azGTrep | 7746-7775 bp | (GT)n | ** | 0.3538 |
| 10 | DEC1az1131 | 7880 bp | G/C | C = 83% / T = 17% | |
| | | | | (48) | |
| 11 | DEC1az1136 | 7885 bp | G/T | G = 67% / T = 32% | |
| | | | | (48) | |
| 12 | DEC1az1140 | 7889 bp | G/A | G = 83% / A = 17% | |
| | | | | (48) | |
| 13 | DEC1az1494 | 8243 bp | A/G | A = 81% / G = 19% | 0.0660 |
| | | | | (48) | |
| 14 | rs1801739 | 8677 bp | A/T | not polymorphic | |
| 15 | rs1058377 | 8905 bp | A/C | not polymorphic | |
| 16 | rs1058380 | 8943 bp | G/A | not polymorphic | |
| 17 | rs1058395 | 9065 bp | A/T | not polymorphic | |

TDTLIKE Terwilliger, (Am J Hum Genet 56:777-787, 1995).
*Data for the distribution of the alleles in caucatians, based on 1220 trio families
(Swedish, German, Italian and British):

| | |
|---|---|
| AT + AC length | Frequency |
| 30 | 31.7% |
| 32 | 26.7% |
| 34 | 20.4% |
| 36 | 5.2% |
| 38 | 1.9% |
| 40 | 13.8% |
| Others (26, 28, 42) | 0.3% |

SEQ ID No:6
AATGCCTCCAAAGACAGGTGGCACCCGTTTTTTTAAATTATTTAATTATATATATA
TATT(AT)₄₋₇(AC)₈₋₁₅ATATATGAGTGTATCTGTCTATCTTTTTAAAAGCATCACCT
Of the most common alleles, 30 appears only to be found as 5xAT + 10AC, 32 is mainly
6xAT + 10xAC, but 5xAT + 11xAC is also found (10-20%) and 34 appears only to be found as
6xAT + 11xAC.

| | |
|---|---|
| **GT length | Frequencies in Caucasians are roughly |
| 13 | 1% |
| 14 | 8% |
| 15 | 58% |
| 16 | 23% |
| 17 | 1% |
| 18 | 6% |
| 19 | 2% |
| 20 | 1% |
| Others (12, 21 & 22) | 0.4% |

SEQ ID No:9
CTGCAAGATTGTTGCATTGTGTATACTGAGATAATCTGAGGCATGGAGAGCA
GATTCAGG(GT)₁₂₋₂₂ATGTGCGTGTGCGTGCACATGTGTGCCTGCGTGTTG
GTATAGGACTTTAAAGCTCCTTTT

TABLE 4

| | |
|---|---|
| Internal | 5′flanking sequence |
| identifier | (SEQ ID Nos: 21 to 37 respectively) |

| | |
|---|---|
| 1 | CCGCGAACAGCGCCGCCGAACTTCCCAACAGGCAGGGAG |
| | AGGGCCGATCCGGGCTGGCAC |
| 2 | GGAGGCGACCGGGGCGCCTCTGCTCCGGAGGGGGCAGAG |
| | GAGGGAGGGAGCGCCGGGCAC |
| 3 | CCTCATCCAGACGCTCCGCTAGTGCAGACAGGAGCGCGC |
| | AGTGGCCCCGGCTCGCCGCGC |
| 4 | CAGGATGTACCCTGCCCACATGTACCAAGTGTACAAGTC |
| | AAGACGGGGAATAAAGCGGAG |
| 5 | AGACAGGTGGCACCCGTTTTTTTAAATTATTTAATTATA |
| | TATATATATTATATATATATA |
| 6 | AATGCCTCCAAAGACAGGTGGCACCCGTTTTTTTAAATT |
| | ATTTAATTATATATATATATT |
| 7 | ATCAGACCCAGCTCCCAAAGTGATGGACTTCAAGGAAAA |
| | ACCCAGCTCTCCGGCCAAAGG |
| 8 | ACTGCGTGCCAGTCATCCAGCGGACTTTCGCTCACTCGA |
| | GTGGGGAGCAGAGCGGCAGCG |
| 9 | CTGCAAGATTGTTGCATTGTGTATACTGAGATAATCTGA |
| | GGCATGGAGAGCAGATTCAGG |
| 10 | CTTTAAAGCTCCTTTTGGCATAGGGAAGTCACGAAGGAT |
| | TGCTTGACATCAGGAGACTTG |
| 11 | AAGCTCCTTTTGGCATAGGGAAGTCACGAAGGATTGCTT |
| | GACATCAGGAGACTTGGGGGG |
| 12 | TCCTTTTGGCATAGGGAAGTCACGAAGGATTGCTTGACA |
| | TCAGGAGACTTGGGGGGGATT |
| 13 | CATAGCACGGTAGTGGTTTGGGGAGGTTTCCGCAGGTCT |
| | GCTCCCCACCCCTGCCTCGGA |
| 14 | CAACGGCATATGGAGTGTCCTTATTGCTAAAAAGGATTC |
| | CGTCTCCTTCAAAGAAGTTTT |
| 15 | TATATAAATATATTAAAAAGGAAAATGTTTCAGATGTTT |
| | ATTTGTATAATTACTTGATTC |
| 16 | TATTTGTATAATTACTTGATTCACACAGTGAGAAAAAAT |
| | GAATGTATTCCTGTTTTTGAA |
| 17 | GAAGGTGTAAAATTGTAAATATTTTTATCTATGAGTAAA |
| | TGTTAAGTAGTTGTTTTAAAA |

TABLE 5

| | |
|---|---|
| Internal | 3′flanking sequence |
| identifier | (SEQ ID Nos: 38-54 respectively) |

| | |
|---|---|
| 1 | CCACGTCCCCGGGGTCTCAGTCCCTGGGTCAACGCAGA |
| | GAGGCTCGCGGTCCGCGGTGCT |
| 2 | GCGCCCGGTATCTTTCTGCGCTTGACTGGCCGGGAGGA |
| | AGGGGGTGACACTGGGGCACCC |
| 3 | ATGGAGCGGATCCCCAGCGCGCAACCACCCCCCGCCTG |
| | CCTGCCCAAAGCACCGGGACTG |
| 4 | GAGGACAGCAAGGTAAGCAAGTGCACCCCTAGGGACCC |
| | TGCGCTCAGCCCCTCGCGCGCG |
| 5 | ACACACACACACACACACACATATATGAGTGTATCTGT |
| | CTATCTTTTTAAAAGCATGACC |
| 6 | TATATGAGTGTATCTGTCTATCTTTTTAAAAGCATGAC |
| | CTACAGACTTAAAACAGCCAAC |
| 7 | TCGGAAGGTCCTGGGAAAAACTGCGTGCCAGTCATCCA |
| | GCGGACTTTCGCTCACTCGAGT |
| 8 | CACGGACACAGACAGTGGCTATGGAGGAGAATCGGAGA |
| | AGGGCGACTTGCGCAGTGAGCA |
| 9 | ATGTGCGTGTGCGTGCACATGTGTGCCTGCGTGTTGGT |
| | ATAGGACTTTAAAGCTCCTTTT |
| 10 | GGGGGATTGTAGCAGACGTCTGGGCTTTTCCCCACCCA |
| | GAGAATAGCCCCCTTCGATACA |
| 11 | ATTGTAGCAGACGTCTGGGCTTTTCCCCACCCAGAGAA |
| | TAGCCCCCTTCGATACACATCA |
| 12 | TAGCAGACGTCTGGGCTTTTCCCCACCCAGAGAATAGC |
| | CCCCTTCGATACACATCAGCTG |
| 13 | GAATAAAGAGAATGTAGTTCCCTACTCAGGCTTTCGTA |
| | GTGATTAGCTTACTAAGGAACT |
| 14 | TTTTTGGTCCAGAGTACTTGTTTTCCCGATGTGTCCAG |
| | CCAGCTCCGCAGCAGCTTTTCA |
| 15 | CACAGTGAGAAAAAATGAATGTATTCCTGTTTTTGAAG |
| | AGAAGAATAATTTTTTTTTTCT |
| 16 | AGAAGAATAATTTTTTTTTTCTCTAGGGAGAGGTACAG |
| | TGTTTATATTTTGGAGCCTTCC |
| 17 | ACTTAATAAAATAATTCTTTTCCTGTGGAAGAGAAAGA |
| | TGGTCTCCTGAGTTTTTTAAAA |

1. A method of identifying a test compound that modulates the expression of BHLHB2 gene comprising:
contacting a cell capable of expressing BHLHB2 gene with a test compound; and
determining the level of expression of the BHLHB2 gene in the presence of the test compound, wherein a decrease or an increase in expression of the BHLHB2 gene, as compared to the level of expression of a BHLHB2 gene in the absence of the compound, is indicative that the test compound modulates BHLHB2 gene expression.

2. A method of identifying a test compound that modulates the activity of a BHLHB2 protein comprising:
contacting the BHLHB2 protein with a test compound; and
determining the level of activity of the BHLHB2 protein in the presence of the compound, wherein a decrease or an increase in BHLHB2 protein activity, as compared to the level of activity of the BHLHB2 protein in the absence of the compound, is indicative that the test compound modulates BHLHB2 protein activity.

3. A method of treating a subject having CAD comprising administering an effective amount of the compound identified in claims 1 or 2.

4. (canceled)

5. A method of treating a patient suffering from CAD comprising administering to said patient an effective amount of an antibody specific for protein BHLBH2.

6. A method of treating a patient suffering from CAD comprising administering to the patient an effective amount of an antisense molecule capable of binding to the mRNA of gene BHLBH2, wherein binding of the antisense molecule to the mRNA causes a decrease in expression of the protein product of gene BHLBH2.

7. A pharmaceutical composition comprising the compounds identified in claims 1 or 2, and a pharmaceutically acceptable adjuvant, diluent or carrier.

8. A method of making a pharmaceutical composition comprising:
contacting a cell capable of expressing BHLBH2 with a test compound;
determining the level of expression of BHLBH2 in the presence of the test compound; wherein a decrease or an increase in expression of BHLBH2, as compared to the level of expression of BHLBH2 in the absence of the compound, is indicative that the test compound modulates BHLBH2 gene expression; and
formulating the test compound that modulates BHLBH2 into a pharmaceutical composition.

9. A method of making a pharmaceutical composition comprising:
contacting a BHLBH2 protein with a test compound;
determining the level of activity of the BHLBH2 protein in the presence of the compound, wherein a decrease or an increase in BHLBH2 protein activity, as compared to the level of activity of the BHLBH2 protein in the absence of the compound, is indicative that the test compound modulates BHLBH2 protein activity; and
formulating the test compound that modulates BHLBH2 protein activity into a pharmaceutical composition.

10. A method for determining if a BHLBH2 gene has an altered level of gene expression comprising:
comparing the level of BHLBH2 gene expression in a cell from a patient having CAD with a control cell; and
determining the level of expression of the BHLBH2 gene in both cells, wherein a decrease or an increase in expression of the BHLBH2 gene, as compared to the level of expression of the BHLBH2 gene in the control cell, indicates that the BHLBH2 has altered gene expression.

11. A method for determining the level of a BHLBH2 protein in a CAD patient compared to a control comprising:
comparing the protein level of BHLBH2 in a cell from a patient having CAD with a control cell; and
determining the level of the BHLBH2 protein in both cells.

12. A method of identifying a binding partner of the BHLHB2 protein comprising:
contacting a BHLHB2 protein with a test target protein; and
determining if the test target protein can interact with the BHLHB2 protein, wherein interaction of the test target protein with BHLBH2 indicates that the test target protein is a BHLBH2 binding partner.

13. The method of claim 12, wherein the method further comprises:
contacting a gene encoding the test target protein with a test compound; and
determining the level of expression of the test target gene in the presence of the test compound, wherein a decrease or an increase in test target gene expression, as compared to the level of expression of the test target gene in the absence of the compound, is indicative that the test compound modulates expression of the test target gene and is useful in the treatment of CAD.

14. The method of claim 12, wherein the method further comprises:
contacting the test target protein with a test compound; and
determining the level of activity of the test target protein in the presence of the test compound, wherein a decrease or an increase in test target protein activity, as compared to the level of activity of the test target protein in the absence of the compound, is indicative that the test compound modulates test target protein activity and is useful in the treatment of CAD.

15. The method of any of claims 13 or 14, wherein the test compound is formulated into a pharmaceutical composition.

16. A method of treating a subject having CAD comprising administering an effective amount of the compound identified in claims 13 or 14.

17. A pharmaceutical composition comprising a compound identified in of any of claims 13 or 14 and a pharmaceutically acceptable adjuvant, diluent or carrier.

18. A method of identifying other components of the CAD biochemical pathway of which BHLHB2 is a component.

19. A method of diagnosing CAD, or a susceptibility thereto in a subject, the method comprising:
determining the level of mRNA of BHLHB2 in a sample from a subject; and
comparing the level of mRNA of BHLHB2 in the sample with a control, wherein a decrease or an increase in the level of mRNA of BHLHB2 in the sample compared to the control indicates that the subject has CAD, or a susceptibility thereto.

20. A method of diagnosing CAD or a susceptibility thereto in a subject, the method comprising:
determining the level of a BHLHB2 protein in a sample from a subject; and
comparing the level of BHLHB2 protein in the sample with a control, wherein a decrease or an increase in the level of the protein in the sample compared to the control indicates that the subject has CAD, or a susceptibility thereto.
